(19) United States
(12) Patent Application Publication
(10) Pub. No.: US 2022/0370797 A1
(43) Pub. Date: Nov. 24, 2022

(54) TECHNIQUES FOR PLACING IMPLANTABLE ELECTRODES TO TREAT SLEEP APNEA, AND ASSOCIATED SYSTEMS

(71) Applicant: Invicta Medical, Inc., Santa Clara, CA (US)

(72) Inventors: Richard W. O'Connor, Atherton, CA (US); Timothy A. Fayram, Gilroy, CA (US); Carl Lance Boling, San Jose, CA (US); Chang Yeul Lee, San Jose, CA (US); Dennis Potts, Scotts Valley, CA (US); Paul Paspa, Santa Clara, CA (US)

(21) Appl. No.: 17/749,025

(22) Filed: May 19, 2022

Related U.S. Application Data
(60) Provisional application No. 63/191,240, filed on May 20, 2021.
(60) Provisional application No. 63/220,355, filed on Jul. 9, 2021.

(51) Int. Cl. A61N 1/36; A61N 1/05; A61N 1/378; A61N 1/375

(52) U.S. Cl. CPC A61N 1/3611; A61N 1/0551; A61N 1/36078; A61N 1/36157; A61N 1/36167; A61N 1/36003; A61N 1/3787; A61N 1/37518

(57) ABSTRACT

Techniques for placing implantable electrodes to treat sleep apnea, and associated devices, systems, and methods are disclosed herein. A representative method includes percutaneously implanting one or more signal delivery devices, each at or near a respective target signal delivery location in a patient. Each signal delivery device can include one or more electrodes, and individual ones of the electrodes can be positioned to produce a net positive protrusive motor response of the patient's tongue. The representative method further includes providing power to one or more of the electrodes from a wearable power source to cause the electrode(s) to deliver an electrical signal to the respective target signal delivery location(s) to produce the net positive protrusive motor response.

### CROSS-REFERENCE TO RELATED APPLICATIONS

The present application claims priority to U.S. Provisional App. No. 63/220,335, filed Jul. 9, 2021, and U.S. Provisional App. No. 63/191,240, filed May 20, 2021, the entireties of which are incorporated herein by reference.

### TECHNICAL FIELD

The present technology is directed generally to techniques for placing implantable electrodes, which are wirelessly coupled to a remote power delivery device, to treat sleep apnea, and associated systems and devices. Representative power delivery devices include a mouthpiece, a device worn in a collar or other neck clothing form factors, and/or an adhesive skin-mounted device.

### BACKGROUND

Obstructive sleep apnea (OSA) is a medical condition in which a patient's upper airway is occluded (partially or fully) during sleep, causing sleep arousal. Repeated occlusions of the upper airway may cause sleep fragmentation, which in turn may result in sleep deprivation, daytime tiredness, and/or malaise. More serious instances of OSA may increase the patient's risk for stroke, cardiac arrhythmias, high blood pressure, and/or other disorders.

OSA may be characterized by the tendency for soft tissues of the upper airway to collapse during sleep, thereby occluding the upper airway. OSA is typically caused by the collapse of the patient's soft palate, oropharynx, tongue, epiglottis, or combination thereof, into the upper airway, which in turn may obstruct normal breathing and/or cause arousal from sleep.

Some treatments have been available for OSA including, for example, surgery, constant positive airway pressure (CPAP) machines, and electrically stimulating muscles or related nerves associated with the upper airway to move the tongue (or other upper airway tissue). Surgical techniques have included procedures to remove portions of a patient's tongue and/or soft palate, and other procedures that seek to prevent the tongue from collapsing into the back of the pharynx. These surgical techniques are very invasive. CPAP machines seek to maintain upper airway patency by applying positive air pressure at the patient's nose and mouth. However, these machines are uncomfortable, cumbersome, and may have low compliance rates.

Some electrical stimulation techniques seek to prevent the tongue from collapsing into the back of the pharynx by causing the tongue to protrude forward (e.g., in an anterior direction) and/or flatten during sleep. However, existing techniques for electrically stimulating the nerves of the patient's oral cavity suffer from being too invasive, and/or not sufficiently efficacious. Thus, there is a need for an improved minimally-invasive treatment for OSA and other sleep disorders.

### BRIEF DESCRIPTION OF THE DRAWINGS

Representative embodiments of the present technology are illustrated by way of example and are not intended to be limited by the Figures, in which like reference numerals generally refer to corresponding parts throughout.

FIG. 1 is a side sectional view depicting a patient's upper airway.

FIG. 2 is a partially schematic, side sectional view of a patient's upper airway, and illustrates elements of a system for treating sleeping disorders in accordance with embodiments of the present technology.

FIG. 3A is a side view of a patient's skull, illustrating representative signal delivery targets in accordance with embodiments of the present technology.

FIG. 3B is a view of a patient's skull, from below, illustrating the hypoglossal nerve and a representative electrode location in accordance with embodiments of the present technology.

FIGS. 3C and 3D illustrate an isometric view and an end view, respectively, of the medial branch of the hypoglossal nerve, and an associated signal delivery device, positioned in accordance with embodiments of the present technology.

FIGS. 4A-4E illustrate an approach for implanting a signal delivery device in accordance with embodiments of the present technology.

FIGS. 5A and 5B are a partially schematic illustration of the ansa cervicalis, hyoglossus, associated musculature, and associated signal delivery devices, positioned in accordance with embodiments of the present technology.

FIGS. 6A-6C are partially schematic illustrations of signal delivery devices configured in accordance with embodiments of the present technology.

FIG. 7A is a representative example of a waveform having waveform parameters selected in accordance with embodiments of the present technology.

FIG. 7B is a representative example of a waveform having active and resting periods in accordance with embodiments of the present technology.

FIG. 8 is a flow diagram illustrating a representative process for implanting and removing a signal delivery device, in accordance with representative embodiments of the present technology.

FIG. 9 is a table illustrating representative equipment used to carry out the process shown in FIG. 8.

FIGS. 10A and 10B illustrate a representative ultrasound probe, and an associated image, in accordance with representative embodiments of the present technology.

FIGS. 11A and 11B illustrate a representative ultrasound probe, and associated image, in accordance with embodiments of the present technology.

FIG. 12 is a schematic illustration of a representative ultrasound probe used for processes in accordance with the present technology.

### DETAILED DESCRIPTION

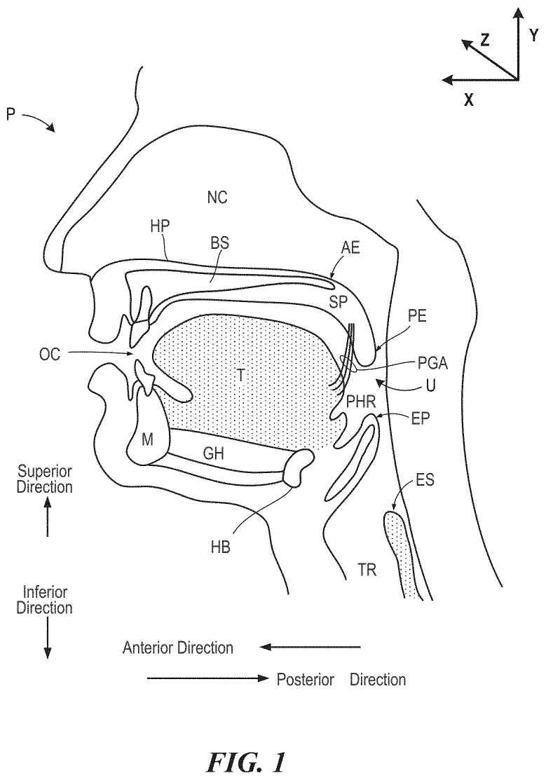

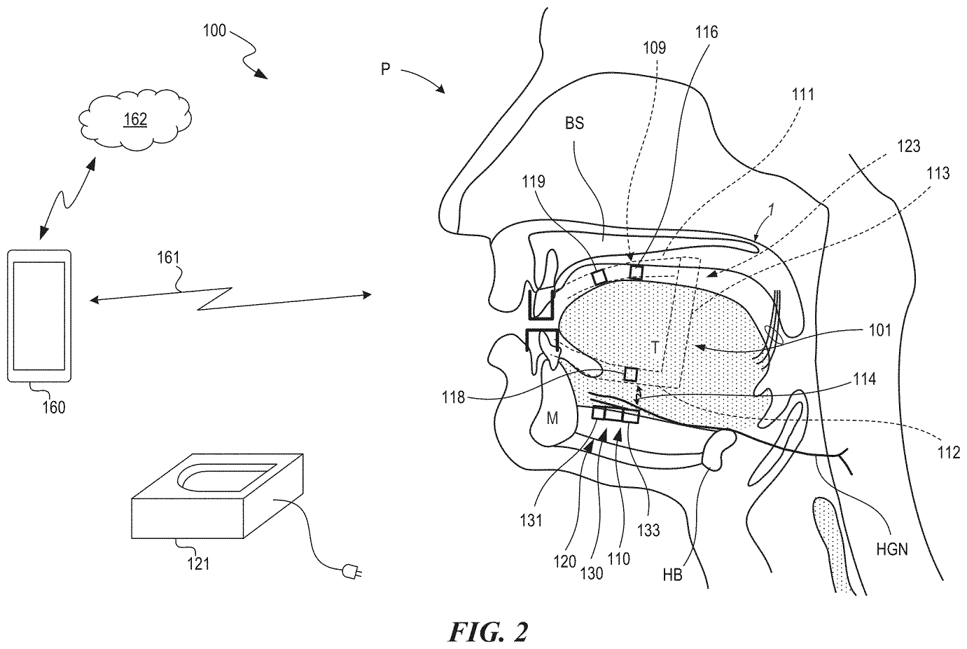

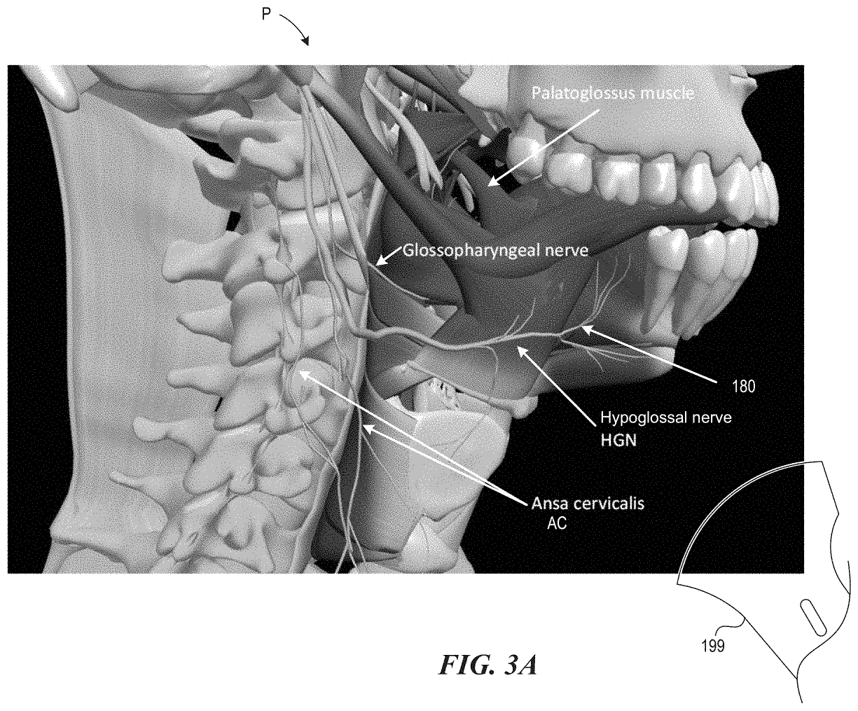

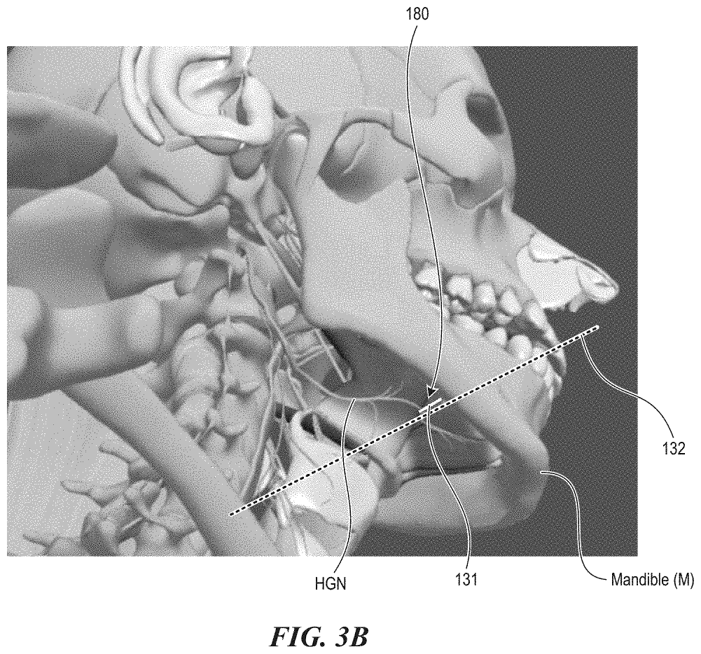

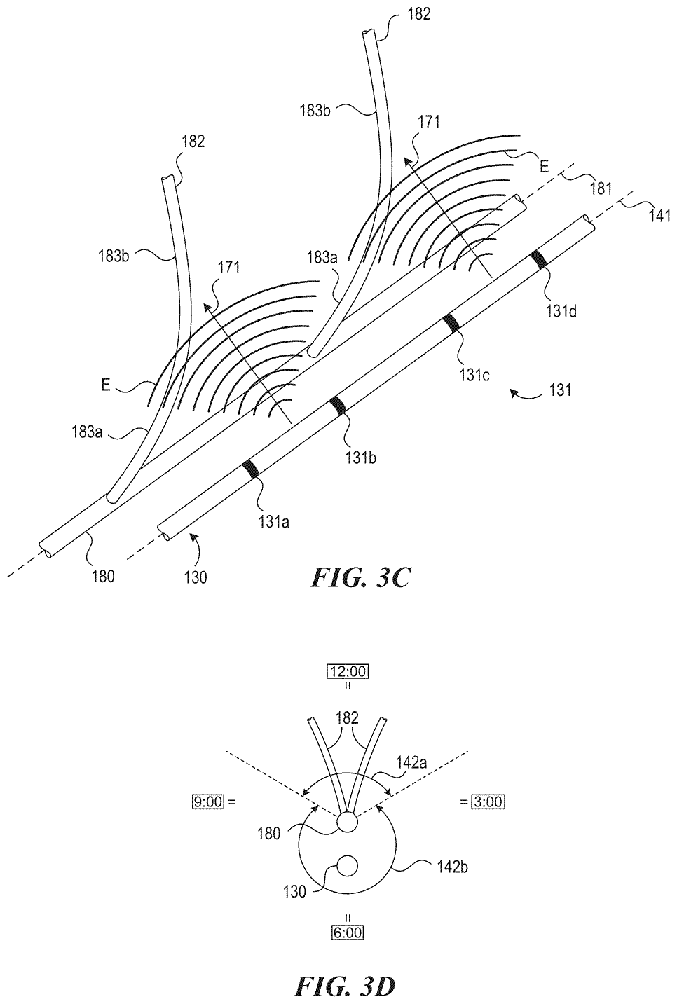

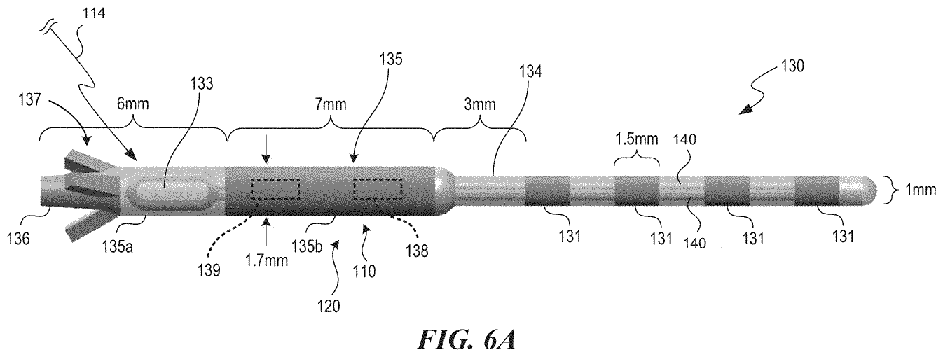

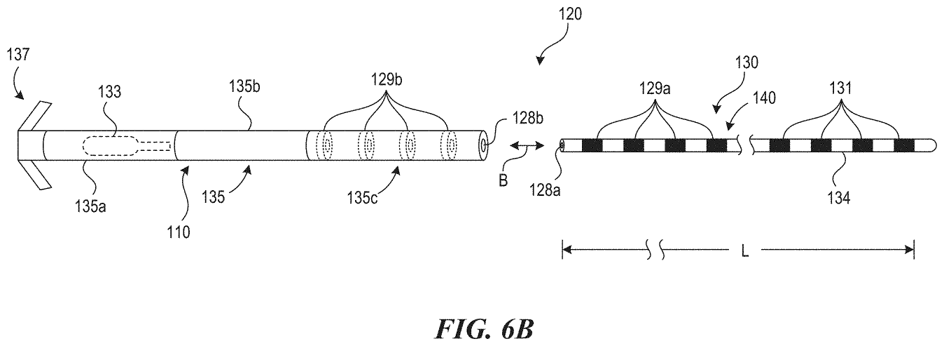

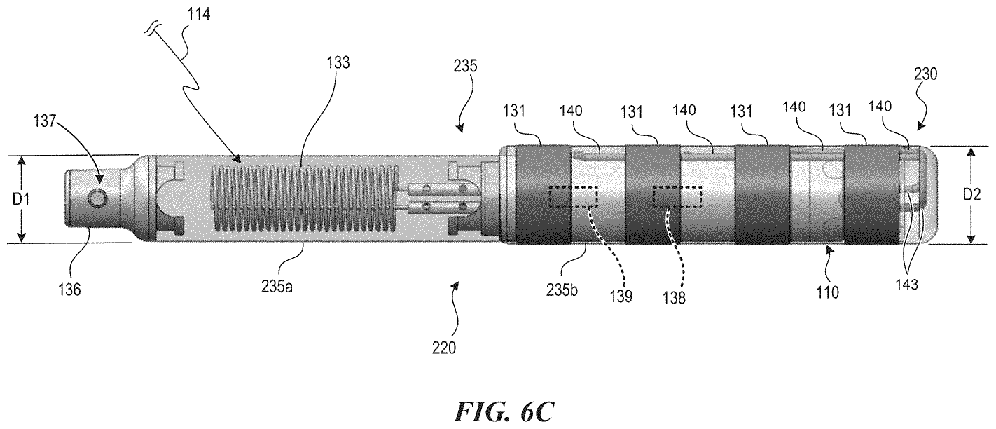

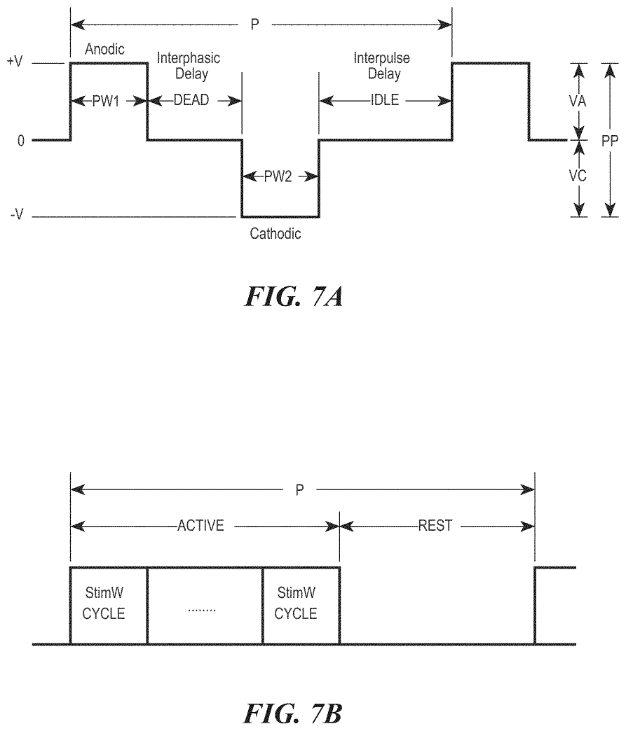

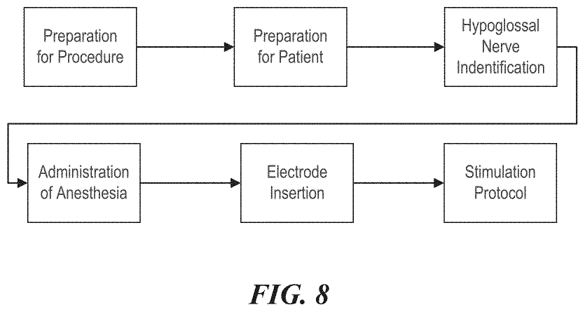

The present technology is discussed under the following headings for ease of readability:
Heading 1: “Introduction”
Heading 2: “Overall Patient Physiology” (with focus on FIG. 1)
Heading 3: “Overall System” (with focus on FIG. 2)
Heading 4: “Representative Stimulation Targets and Implant Techniques” (with a focus on FIGS. 3A-5B)
Heading 5: “Representative Signal Delivery Devices” (with a focus on FIGS. 6A-6C)
Heading 6: “Representative Waveforms” (with a focus on FIGS. 7A and 7B)
Heading 7: “Further Implant Techniques” (with a focus on FIGS. 8-12)

While embodiments of the present technology are described under the selected headings indicated above, other embodiments of the technology can include elements discussed under multiple headings. Accordingly, the fact that an embodiment may be discussed under a particular heading does not necessarily limit that embodiment to only the elements discussed under that heading.

### 1. Introduction

Electrical stimulation for obstructive sleep apnea (OSA) typically includes delivering an electrical current that modulates nerves and/or muscles, e.g., to cause the tongue and/or other soft tissue to move. The electrical stimulation can accordingly remove an obstruction of the upper airway, or prevent the tongue or other soft tissue from collapsing or obstructing the airway. As used herein, the terms “modulate” and “stimulate” are used interchangeably to mean having an effect on, e.g., an effect on a nerve and/or or a muscle that in turn has an effect on one or more motor functions, e.g., a breathing-related motor function.

Representative methods and apparatuses for reducing the occurrence and/or severity of a breathing disorder, such as OSA, are disclosed herein. In accordance with representative embodiments, a minimally-invasive signal delivery device is implanted proximate to or adjacent to nerves that innervate the patient's oral cavity, soft palate, oropharynx, and/or epiglottis. Representative nerves include the hypoglossal nerve, branches of the ansa cervicalis and/or the vagus nerves, which are located adjacent and/or around the oral cavity or in the neck. The signal delivery device can be implanted in the patient via a percutaneous injection. A non-implanted power source, e.g., including one or more mouthpiece portions, collar portions, chinstrap portions, pillow portions, mattress overlay portions, other suitable “wearables,” and/or one or more adhesive, skin-mounted devices, can wirelessly provide electrical power to the implanted signal delivery device. The signal delivery device emits accurately targeted electrical signals (e.g., pulses) that improve the patient's upper airway patency and/or improve the tone of the tissue of the intraoral cavity to treat sleep apnea. The electrical current delivered by the signal delivery device can stimulate at least a portion of a patient's hypoglossal nerve and/or other nerves associated with the upper airway. By moving the tongue forward and/or by preventing the tongue and/or soft tissue from collapsing onto the back of the patient's pharynx, and/or into the upper airway, the devices and associated methods disclosed herein can in turn improve the patient's sleep, e.g., by moving the potentially obstructing tissue in the upper airway/pharynx down. More specifically, applying the electrical signal to the medial branch of the hypoglossal nerve can cause the tongue to move forward (anteriorly), and applying the electrical signal to the ansa cervicalis can cause the hyoid bone, the thyroid (e.g., the thyroid cartilage), and/or the larynx to move downward (inferiorly or caudally), a motion typically referred to as caudal traction.

Many embodiments of the technology described below may take the form of computer- or machine- or controller-executable instructions, including routines executed by a programmable computer or controller. Those skilled in the relevant art will appreciate that the technology can be practiced on computer/controller systems other than those shown and described below. The technology can be embodied in a special-purpose computer, controller or data processor that is specifically programmed, configured or constructed to perform one or more of the computer-executable instructions described below. Accordingly, the terms “computer” and “controller” as generally used herein refer to any suitable data processor and can include Internet appliances and hand-held devices (including palm-top computers, wearable computers, tablets, cellular or mobile phones, multi-processor systems, processor-based or programmable consumer electronics, network computers, mini computers and the like). Information handled by these computers can be presented at any suitable display medium, including a liquid crystal display (LCD).

The present technology can also be practiced in distributed environments, where tasks or modules are performed by remote processing devices that are linked through a communications network. In a distributed computing environment, program modules or subroutines may be located in local and remote memory storage devices. Aspects of the technology described below may be stored or distributed on any suitable computer-readable media, including one or more ASICs, (e.g., with addressable memory), as well as distributed electronically over networks. Data structures and transmissions of data particular to aspects of the technology are also encompassed within the scope of the embodiments of the technology.

### 2. Overall Patient Physiology

Representative embodiments described herein include signal delivery devices having electrodes that can be positioned to deliver one or more electrical currents to one or more specific target locations, e.g., specific nerves and/or specific positions along a nerve. FIG. 1 illustrates the general anatomy of the patient's oral cavity, and later Figures illustrate specific target locations. Such locations include locations along the patient's hypoglossal nerve, branches of the ansa cervicalis, and/or vagus nerves, as those nerves that innervate muscles of airway (e.g., palatal, oropharyngeal, laryngeal, omohyoid, sternohyoid, and/or sternothyroid muscles) besides the tongue. The target location can be identified with respect to any of, or any combination of, intrinsic or extrinsic muscles, associated nerve branches and/or portions thereof, and/or other physiological features. Such a target location and/or position can also be distal from the salivary glands (e.g., medial to the sublingual salivary gland) and/or other structures to avoid causing pain and/or other undesired effects.

FIG. 1 illustrates a patient P relative to a coordinate system in which the x-axis denotes the anterior-posterior directions, the y-axis denotes the superior-inferior directions, and the z-axis denotes the medial-lateral directions. The patient P has a hard palate HP which overlies the tongue T and forms the roof of the oral cavity OC (e.g., the mouth). The hard palate HP includes bone support BS, and thus does not typically deform during breathing. The soft palate SP, which is made of soft tissue such as membranes, fibrous material, fatty tissue, and muscle tissue, extends rearward (e.g., in a posterior direction) from the hard palate HP toward the back of the pharynx PHR. More specifically, an anterior end AE of the soft palate SP is anchored to a posterior end of the hard palate HP, and a posterior end PE of the soft palate SP is unattached. Because the soft palate SP does not contain bone or hard cartilage, the soft palate SP is flexible and may collapse onto the back of the pharynx PHR and/or flap back and forth (e.g., especially during sleep).

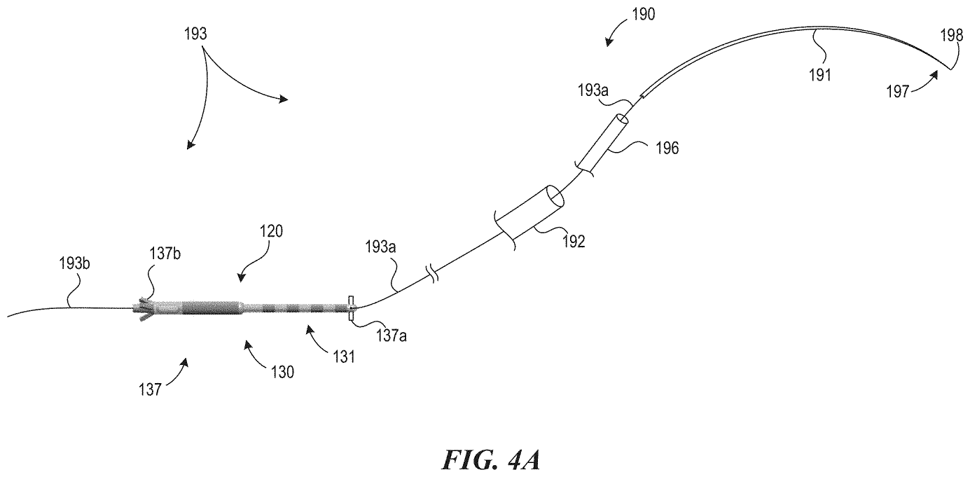

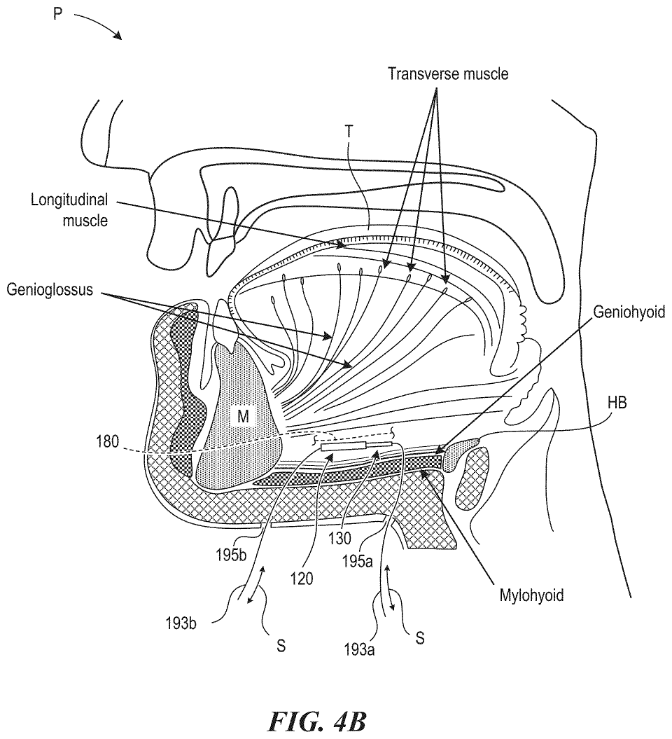

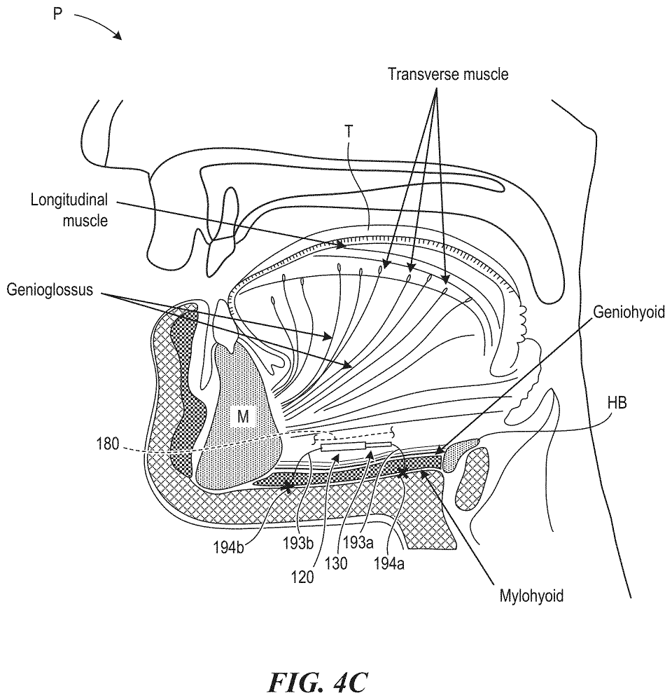

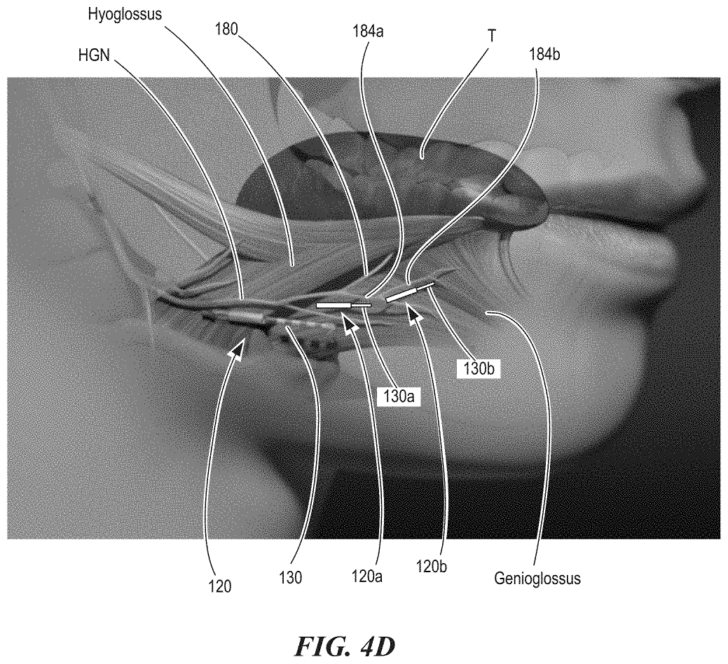

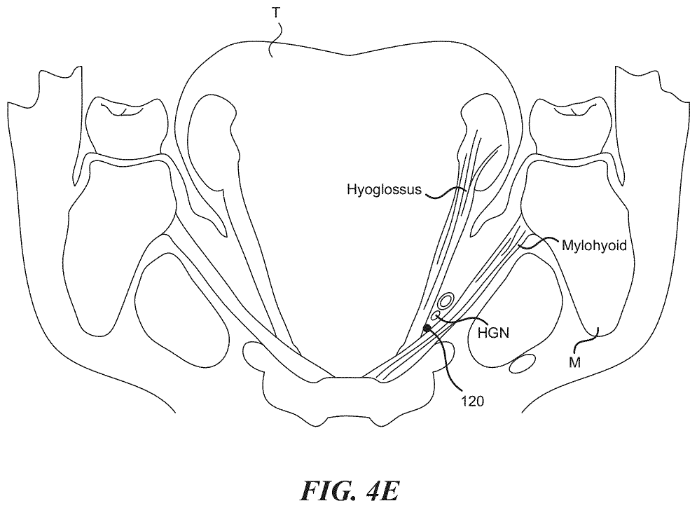

The pharynx PHR, which passes air from the oral cavity OC and the nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the palatoglossal arch PGA, which runs downward on either side to the base of the tongue T. Although not shown for simplicity, the pharynx PHR includes the nasopharynx, the oropharynx, and the laryngopharynx. The nasopharynx lies between an upper surface of the soft palate SP and the wall of the throat (i.e., superior to the oral cavity OC). The oropharynx lies behind the oral cavity OC, and extends from the uvula U to the level of the hyoid bone HB. The oropharynx opens anteriorly into the oral cavity OC. The lateral wall of the oropharynx includes the palatine tonsil, and lies between the palatoglossal arch PGA and the palatopharyngeal arch. The anterior wall of the oropharynx includes the base of the tongue T and the epiglottic vallecula. The superior wall of the oropharynx includes the inferior surface of the soft palate SP and the uvula U. Because both food and air pass through the pharynx PHR, a flap of connective tissue called the epiglottis EP closes over the glottis (not shown for simplicity) when food is swallowed to prevent aspiration. The laryngopharynx is the part of the throat that connects to the esophagus ES, and lies inferior to the epiglottis EP. Below the tongue T is the lower jaw or mandible M, and the geniohyoid muscle GH, which is one of the muscles that controls the movement of the tongue T. The genioglossus muscle, which also controls tongue movement, and is a particular target of the presently disclosed therapy, is discussed later with reference to FIG. 4B.

### 3. Overall System

FIG. 2 is a partially schematic, isometric illustration of a system 100, shown in the context of the patient's anatomy, in a view similar to that described above with reference to FIG. 1. In a representative embodiment, the system 100 includes both implanted elements and external elements. The implanted elements can include one or more implantable devices 120. Each implantable device 120 can include a signal delivery device 130 positioned adjacent to the target neural and/or muscle structure. The signal delivery device 130 can be secured in place with anchors, suture threads, and/or other devices. The signal delivery device anchors can include, for example, one or more tines, helices, mesh coverings, expanding stents, and the like. The signal delivery device 130 is operatively coupled to a signal generator 110. In some embodiments, all the signal generation functions are performed by the implantable device 120, and in other embodiments, some signal generation functions may be performed by external elements. The signal generation functions and signal delivery functions may be performed by a single implantable device 120, or multiple devices.

The system 100 can further include a wearable device 101 that carries a power source 109. For purposes of illustration, the wearable device 101 is shown in FIG. 2 as including an intraoral device 123, e.g., a mouthpiece, that in turn carries the power source 109. As indicated above, the wearable device 101 can have other suitable configurations (e.g., collar, chinstrap, pillow, mattress overlay, among others) in other embodiments. The power source 109 provides power to a signal generator 110, which generates and directs signals (e.g., therapy signals) to one or more electrodes 131 carried by a signal delivery device 130. The signal delivery device 130 can be implanted at or proximate to a target nerve, such as the patient's hypoglossal nerve HGN, using a minimally invasive technique, e.g., using a percutaneous injection needle, as will be described later under Heading 4. The power source 109 provides power to the signal generator 110 via a wireless power transmission link 114, for example, an RF transmission link configured to wirelessly provide power at any of the frequencies and/or frequency ranges described later under Heading 5, and/or any other suitable frequency/frequency range.

Elements carried by the wearable device 101, and (directly or indirectly) the implantable device 120, can be controlled by a programmer 160, via a wireless programmer link 161. In addition, the programmer 160 can communicate with the cloud 162 and/or other computer services to upload data received from the patient P, and/or download information to the wearable device 101 and/or the implantable device(s) 120. Downloaded data can include instructions and/or other data regarding suitable treatments (e.g., from other similarly-situated patients), updates for software executed on the circuitry carried by the wearable device 101 and/or the implantable device(s) 120, and/or other useful information. In other embodiments, the implantable device(s) 120 and/or the wearable device 101 include state machine components, which are not updatable. Representative downloaded data received from the patient can include respiratory rate, heart rate, audio signals (corresponding to audible snoring, hypopnea events, and/or apnea events), body temperature, head orientation/position, saturated blood oxygen levels, air flow levels, thyroid movement, and/or tongue movement, among others. In any of the foregoing embodiments, the wearable device 101 transmits power to the implantable devices 120 via the one or more power transmission links 114, and receives power (e.g., on an intermittent basis) from a charger 121. The charger 121 can accordingly include a conventional inductive coupling arrangement (e.g., Qi standard charging) and/or a conventional wired connection.

In order to fit comfortably, the wearable device 101 (whether an intraoral device 123 or other type of wearable) can be custom-fit to the patient, or can be made available in different sizes, and/or can be partially configurable to fit individual patients. The intraoral device 123 is particularly suitable when the associated signal delivery device 130 is positioned at or proximate to target neural populations (e.g., the HGN) within the oral cavity. Further details of representative intraoral devices are disclosed in pending U.S. application Ser. No. 17/518,414, filed Nov. 3, 2021, the entirety of which is incorporated herein by reference. Whether the wearable device has a mouthpiece form factor or another suitable form factor, it can provide power to the implantable device 120, even if the implantable device is used to target neural populations other than, and/or in addition to, the HGN, e.g., branches of the vagus and/or ansa cervicalis nerves. In still further embodiments, the power source 109 can be mounted to the patient's skin via an adhesive, though it is expected that avoiding an adhesive will be more desirable/effective for the patient.

With reference to the specific embodiment shown in FIG. 2, the intraoral device 123 can include both an upper mouthpiece portion 111, and a lower mouthpiece portion 112. The two mouthpiece portions 111, 112 can be coupled together via a connector 113. The connector 113 can provide a wired communication link between the two mouthpiece portions, and/or the connector 113 can mechanically position (and/or maintain the position of, or stabilize) the lower mouthpiece portion 112 relative to the upper mouthpiece portion 111. This approach can be used to, for example, advance the patient's lower jaw or mandible M relative to the patient's upper jaw, which is indicated by the bone structure BS in FIG. 2. For example, embodiments of the present technology avoid or at least reduce jaw laxity (the patient's mouth hanging agape) using physical elements of the wearable device 101, in addition to the electrical stimulation powered by the wearable device. For example, a wearable device that includes a collar and/or chin strap can mechanically stabilize the patent's jaw in a target position.

The power source 109 can include one or more charge storage devices 116 (e.g., one or more batteries) that receive power from the charger 121 and store the power for transmission to the signal implantable device 120. Accordingly, the power source 109 can include circuitry (e.g., first circuitry) that receives power from the charge storage device 116, conditions the power (e.g., converts the power from DC to an RF waveform), and transmits the power to a power transmission antenna 118. The power transmission antenna 118 in turn transmits the power to the implantable device 120 via the wireless power transmission link 114 (e.g., an RF transmission link) and an electrode receiver antenna 133 carried by the signal delivery device 130.

The intraoral device 123 can further include a data transceiver antenna that receives data from the programmer 160, and/or transmits data to the programmer 160. Data transmitted to the programmer 160 can include sensor data obtained from one or more sensor(s) 119. Accordingly, the intraoral device 123 can carry the functional elements/components required to direct power to the signal delivery device 130, and can communicate with the programmer 160 so as to provide effective therapy for the patient.

### 4. Representative Stimulation Targets and Implant Techniques

Several stimulation targets and implantation techniques are described and/or illustrated with reference to FIGS. 3A-5. For the purpose of illustrative clarity, these stimulation targets and implantation techniques are shown with reference to a left or right side of the patient P's anatomy, for example, a left medial branch of a left hypoglossal nerve of the patient P. It will be appreciated, however, that at least some or all of the stimulation targets and/or implantation techniques described and/or illustrated with reference to FIGS. 3A-5 are equally suitable for application to another side of the patient's anatomy, for example, a right medial branch of a right hypoglossal nerve of the patient P. Additionally, at least some of the stimulation targets and/or implantation techniques can be used for bilateral signal delivery, for example, to apply a first electrical signal to a first stimulation target on a first side of the patient P and to apply a second electrical signal to a second stimulation target on a side of the patient P. In some embodiments, the first and second stimulation targets can be corresponding left and right portions of the patient's anatomy, such as the left and right medial branches of the left and right hypoglossal nerves. In other embodiments, the first and second stimulation targets can be different, such as a left medial branch of the left hypoglossal nerve and a right ansa cervicalis nerve of the patient.

FIG. 3A is a partially schematic, partially cut-away sagittal view of the neck and lower head region of the patient P. FIG. 3A illustrates representative neural structures of this region, including the hypoglossal nerve HGN (and its medial branch 180) and the ansa cervicalis AC. FIG. 3A also illustrates a representative ultrasound probe 199, used to aid in the process of positioning electrodes, which direct therapy signals to the target nerves.

FIG. 3B is a partially schematic, isometric illustration of the patient's skull, looking upwardly toward the mandible M. FIG. 3B also illustrates the hypoglossal nerve HGN which innervates the muscles controlling the patient's tongue T (FIG. 1). In representative embodiments, one or more electrodes 131 are positioned along the hypoglossal nerve HGN, in particular, at the medial branch 180 of the HGN, in an electrode plane 132 defined by the medial branch 180. By precisely positioning the electrode(s) 131 within this plane 132, and adjacent to the hypoglossal nerve HGN, it is expected that systems in accordance with embodiments of the present technology can more effectively control the patient's airway patency, without causing discomfort, and/or other undesirable effects, and/or in a manner that reduces the amount of power required to produce effective therapy signals. As discussed elsewhere herein, other representative target nerves include the ansa cervicalis and vagal nerves, and/or one or more of the muscles innervated by these nerves. Still further representative targets include cranial nerves (e.g., the glossopharyngeal nerve) and the palatoglossus muscle, which are shown in FIG. 3A, and the left and/or right phrenic nerves.

FIG. 3C is a partially schematic illustration of the medial branch 180, and an associated signal delivery device 130, positioned in accordance with embodiments of the present technology. The medial branch 180 extends along a nerve axis 181 and innervates oral cavity muscles such as the genioglossus and geniohyoid muscles, which tend to pull the tongue forward (anteriorly), thus reducing the tendency for the soft tissue of the palate to prolapse into the patient's airway. However, the medial branch 180 also includes retrusers 182 which innervate muscles such as the styloglossus and the hyoglossus muscles, which tend to pull the soft tissue backward (posteriorly), and/or can cause the tongue to curl left or right within the mouth—both are motor responses that can obstruct the patient's airway. Accordingly, it can be advantageous to stimulate the medial branch 180 via the electrodes 131 in a manner that results in a net positive protrusive effect or a net protrusive motor response. This can include, for example, stimulating the medial branch 180 so as to avoid activating the retrusers 182 entirely. Additionally, or alternatively, the net positive protrusive effect can be obtained when the protrusive response to an electrical signal is greater than, or otherwise counteracts, the retrusive response to the electrical signal. This can include, for example, delivering an electrical signal to one or more of a patient's nerves and/or muscles such that, in response to the electrical signal, the patient's airway is more open and/or allows more airflow than when the electrical signal is not delivered. One approach for obtaining the net positive protrusive effect is to position the electrodes 131 to preferentially stimulate the medial branch 180 itself, without stimulating (or without significantly stimulating) the retrusers 182.

As shown in FIG. 3C, the retrusers 182 typically include a first portion 183a that extends parallel or at least partially parallel to the nerve axis 181 of the medial branch 180. The retrusers 182 further include a second portion 183b that bends away from the nerve axis 181. Accordingly, one approach for avoiding or reducing stimulation of the retrusers 182 is to position the electrodes 131 axially so that the corresponding electrical fields they produce are less likely to activate the retrusers 182. As shown in FIG. 3C, the electrodes 131 are arranged in electrode pairs, including a first pair (comprising first and second electrodes 131a and 131b), and a second pair (comprising third and fourth electrodes 131c, 131d). Other embodiments can include more or fewer electrodes and/or electrode pairs. Each electrode pair generates an electrical field E which decreases in strength in a direction away from the electrodes 131, as indicated by decreasing field strength arrows 171. The electrical fields E preferentially activate neural tissue that extends transverse to the field, rather than parallel to the field. Accordingly, with a device axis 141 of the signal delivery device 130 generally parallel to the nerve axis 181 of the medial branch 180, the electrical fields E preferentially activate the medial branch 180. However, if the electrical fields E are positioned close to the first portions 183a of the retrusers 182 (which are also parallel or close to parallel to the nerve axis 181), then the electrical fields E may also activate the retrusers 182. One approach for avoiding this outcome is to position the electrodes 131 to be offset along the nerve axis 181 relative to the first portions 183a of the retrusers 182. In this way, the electrical field E is less likely to activate the retrusers 182 at the first portions 183a. Although the second portions 183b of the retrusers 182 are transverse to the electrical field (and therefore potentially susceptible to the field), the field at the second portions 183b is expected to be too weak to have a significant effect on the retrusers 182. In these and other embodiments, one or more of the electrodes 131 can be masked (e.g., circumferentially masked), segmented (e.g., circumferentially segmented, individually addressable), directional, at least partially covered, and/or otherwise configured to direct the electrical field in specific direction(s) to further reduce the likelihood of stimulating the retrusers 182.

Another approach for reducing the effect of the electrical fields on the retrusers 182 is to selectively position the electrodes circumferentially, as illustrated in FIG. 3D. As shown in FIG. 3D, the retrusers 182 tend to exit the medial branch 180 in a generally superior direction, while the signal delivery device 130 is positioned inferior to the medial branch 180. Accordingly, if the retrusers 182 extend away from the medial branch 180 in a first area 142a at a clock position of from about 10 o'clock to about 2 o'clock (measured clockwise), the signal delivery device 130 can be positioned in a second area 142b away (e.g., axially offset, opposite, and the like) from the first area 142a: i.e., between about 2 o'clock and about 10 o'clock (measured clockwise), and/or any suitable subarea therein (e.g., between any of 2 o'clock, 3 o'clock, 4 o'clock, 5 o'clock, 6 o'clock, 7 o'clock, 8 o'clock, 9 o'clock, and 10 o'clock). If the retrusers 182 extend in a generally inferior direction from the medial branch 180, the signal delivery device 130 can be positioned generally superior to the medial branch 180.

A further approach for reducing the effect(s) of the electrical fields on the retrusers 182 is to position the electrodes at or proximate to the motor end plate of the target nerve, such as where the HGN innervates the patient's tongue and/or at or within the genioglossus muscle(s). For example, the signal delivery device 130 can be positioned proximate to and/or adjacent to a brachiated portion of the patient's target nerve. This is described in further detail with reference to FIG. 4D. With the signal delivery device 130 in this position, the electrical field E generated by the signal delivery device 130 is spaced apart from the retrusers 182 and is expected to be too weak to have a significant effect on the retrusers 182. In some aspects, positioning the signal delivery device 130 further anterior, such as further into the brachiated portion of the patient's target nerve, can further focus the electrical fields on the target nerve and/or further reduce the likelihood of stimulating the retrusers 182. The foregoing techniques (axial location, circumferential “clocking,” and brachial positioning) can be used either individually or in combination, and it is expected that using these techniques in combination will further reduce the likelihood for activating the retrusers 182.

As indicated above, it can be important to carefully position the electrodes to enhance the beneficial effects associated with the electrical therapy, and reduce counter effects, such as activating the retrusers 182. Example A, discussed under Heading 7, discloses a technique for percutaneously introducing and positioning a signal delivery device via a single entry location, with the aid of an ultrasound probe (shown in FIG. 3A).

In one approach a stylet is used to form a single puncture in the patient's skin. The puncture can be located in a posterior submandibular region of the patient. The signal delivery device 130 can be percutaneously introduced (e.g., implanted, injected, and/or the like) through the posterior submandibular puncture and be positioned proximate the medial branch 180 of the hypoglossal nerve HGN.

In another approach a stylet is used to form a single puncture in the patient's mouth. The puncture can be located in an intraoral sublingual region of the patient's mouth, such as under the ventral surface of the tongue in the floor of the mouth, posterior to the sublingual caruncle and angled inferolaterally towards the medial branch of hypoglossal nerve. The signal delivery device 130 can be percutaneously introduced through the intraoral sublingual puncture and be positioned proximate the medial branch 180 of the hypoglossal nerve HGN.

Another approach, described below with reference to FIGS. 4A-4C, uses a stylet and two punctures in the patient's skin to position the signal delivery device 130. The stylet can be curved, straight, or have any other suitable configuration. In particular embodiments, the signal delivery device can include a suture thread at each end, so that the practitioner can pull on one end and/or the other to precisely locate the signal delivery device (and the electrodes it carries) at the target location.

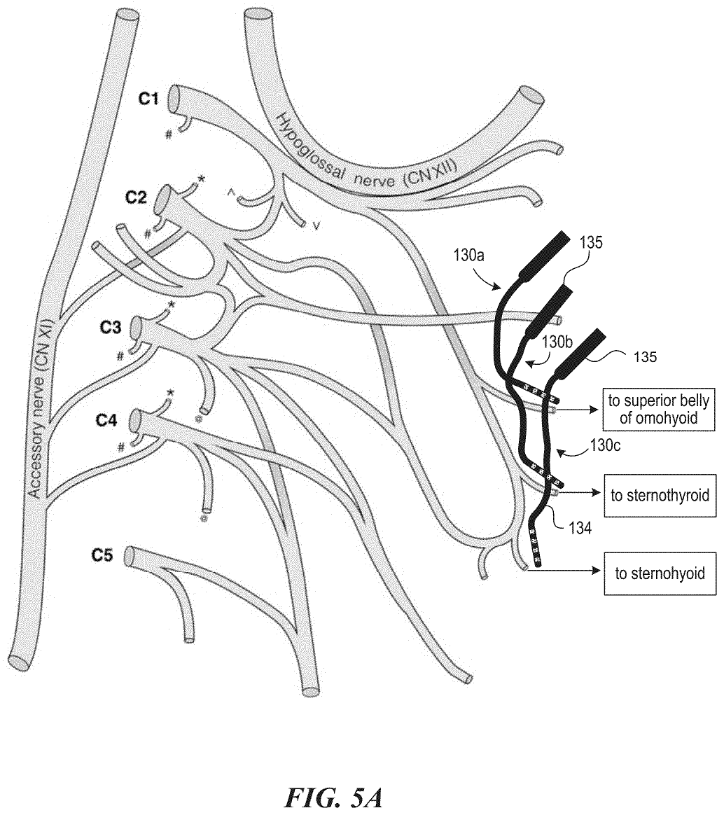

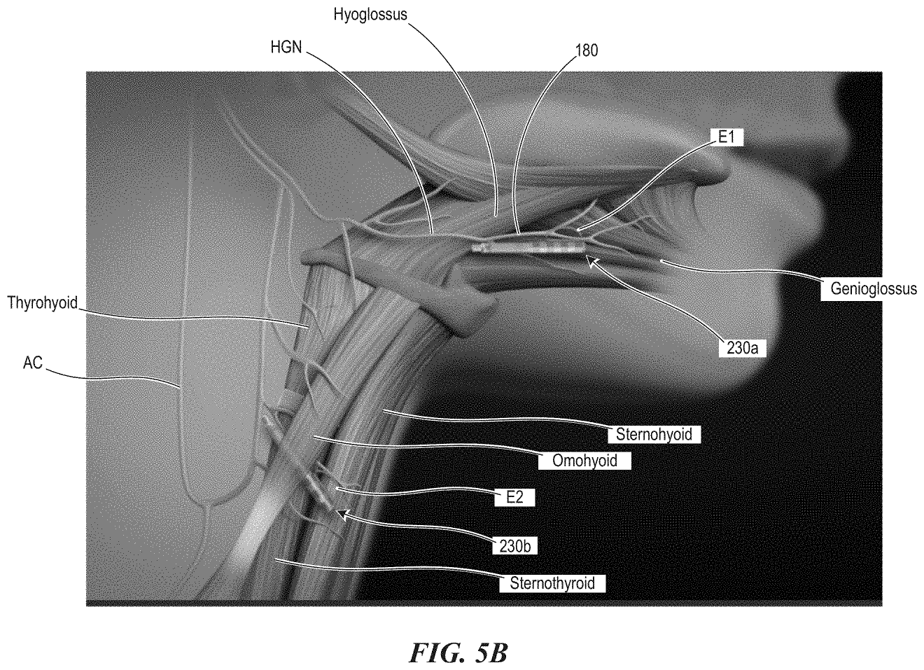

FIG. 4A illustrates a representative set of implant tools 190 used to implant an implantable device 120 in accordance with embodiments of the present technology. The implantable device 120 includes the signal delivery device 130, which in turn includes electrodes 131 that provide electrical stimulation to the target neural population. In some embodiments, such as shown in FIGS. 5A, 6A, and 6B, the signal delivery device 130 includes a lead 134 that carries the electrodes 131. In other embodiments, such as shown in FIGS. 5B and 6C, the lead 134 can be omitted, and the signal delivery device 130 can be “leadless” and/or carry the electrodes 131 on an exterior surface of the signal delivery device 130, such that one or more components of the signal delivery device 130 can be positioned within (e.g., radially inwardly from, or in the annulus of) one or more of the electrodes 131. One end of the implantable device 120 is attached to a proximal suture thread 193a, and the opposite end is attached to a distal suture thread 193b (referred to collectively as suture threads 193). The implantable device 120 can further include one or more anchors 137, shown as a proximal anchor 137a, and a distal anchor 137b. In at least some embodiments, the anchors 137 can be eliminated due to the implantable device 120 being held in place via both the proximal and distal suture threads 193a, 193b.

The proximal suture thread 193a is attached to a curved needle 191. Depending upon the dimensions of the implantable device 120, the implant tools can further include a dilator 196, an introducer 192, which can include a cannula through which the implantable device 120 can be positioned within the patient, and/or other percutaneous insertion device(s) configured to facilitate directing the implantable device 120 into the opening formed by the needle 191, such as via the Seldinger method. For example, the introducer 192 can form a percutaneous insertion pathway through the patient's skin and through which the implantable device 120 can be percutaneously inserted, implanted, injected, and/or the like. Whether the needle 191 is curved (as shown in FIG. 4A) or straight (as may be the case in other embodiments), the needle can have a diameter in a range of from 20 gage to 10 gage, or 18 gage to 12 gage in particular embodiments. The dilator 196 can have a diameter in a range of from 3 Fr to 12 Fr (1 mm to 4 mm). The needle 191 and/or introducer 192 can be initially inserted at a relatively steep trajectory angle (e.g., 60° relative to the skin surface), and then swung down toward the skin to a more shallow angle (e.g., 20° relative to the skin surface) to align the needle with the HGN. Adjusting the insertion trajectory of the needle 191 (and/or other percutaneous insertion device(s), such as the introducer 192) once a portion of the needle 191 is percutaneous can avoid contacting the needle 191 with other structures (e.g., the mandible) along the insertion trajectory and/or reduce or eliminate the likelihood of penetrating other, non-target portions of the patient's anatomy (e.g., salivary glands, vasculature, nerves, mandible bones, and the like). The need for changing the approach angle can be reduced or eliminated when the needle 191 is curved, for example, as described above.

In some embodiments, the needle 191 and/or another percutaneous insertion device can be configured to stimulate the patient's tissues during insertion. For example, as shown in FIG. 4A, the needle 191 can include one or more electrodes 197 positioned at or proximate a terminus 198 of the needle 191. The precise location of the needle can be identified by delivering electrical stimulation to the patient via the needle and observing the patient's motor response. The practitioner can use ultrasound and/or another suitable visualization technique, in addition to or in lieu of inducing a motor response. Accordingly, in at least some embodiments, the practitioner can use a combination of visual navigation and stimulation-response navigation to precisely align the needle with the HGN (or other target nerve) such that, when the implantable device 120 is introduced, the implantable device 120 is expected to be closer to and/or more closely aligned with the HGN. In some embodiments, a practitioner can use stimulation-response navigation to identify the needle's position when operating in portions of the patient's anatomy in which the needle 191 is difficult to visualize (using, e.g., ultrasound), such as proximate to/within the brachiation of the HGN.

Depending on the embodiment, the foregoing elements can be removed axially, or can be pre-slitted and peeled off. In operation, the needle 191 is directed into the patient's tissue at a first point, forming a first opening. The needle can exit the patient's tissue at a second point, forming a second opening. The practitioner can then pull the implantable device 120 through the first opening via the needle 191, and use the proximal and distal suture threads 193a, 193b to more precisely locate implantable device 120 within the patient. Additionally, or alternatively, the needle 191 can be hollow such that the implantable device 120 can be positioned within the patient by inserting the implantable device 120 through the needle 191 and percutaneously into the patient, with or without using the suture threads 193a, 193b, and/or via a single opening. In these and other embodiments, one or more other percutaneous insertion devices, such as the introducer 192, the dilator 196, and/or a cannula, can be inserted over the needle 191 to assist with the percutaneous insertion of the implanted device 120. For example, the needle can be used to stimulate tissue to identify an implant site and facilitate placement of one or more dilators and/or cannulas over the needle, such that the needle can be used to position a cannula configured to deliver the implantable device to the implant site. In these and other embodiments, the needle 191 can optionally include a lumen and/or an atraumatic tip. In at least some embodiments, the needle can be configured to operate as a dilator and deliver a cannula directly, such that the dilator 196 can be omitted.

FIG. 4B is an enlarged view of the patient's lower jaw, illustrating the longitudinal and transverse muscles of the tongue T, as well as the genioglossus, geniohyoid, and mylohyoid muscles. The implantable device 120 is shown after it has been inserted into the patient P via the needle 191 (FIG. 4A), so as to be positioned inferior to the genioglossus at the intersection of the genioglossus and the geniohyoid muscles. The signal delivery device 130 is also adjacent to the medial branch 180, which is shown schematically in dashed lines. The needle 191 was introduced into the patient by forming a distal opening 195b, and exited the patient at a proximal opening 195a. In other embodiments, the needle 191 can be introduced into the patient via the proximal opening 195a and exit the patient via the distal opening 195b. Although in FIG. 4B both the proximal opening 195a and the distal opening 195b are illustrated as being formed in the patient's submandibular space, in other embodiments the proximal opening 195a and/or the distal opening 195b can be formed intraorally, sublingually, and/or in any other suitable position. In at least some embodiments, for example, the needle 191 can be introduced into the patient via a submandibular opening and exit the patient via an intraoral sublingual opening.

After the needle 191 and any dilators or introducers have been removed, the remaining proximal suture thread 193a and distal suture thread 193b extend out from the patient P at the proximal opening 195a and the distal opening 195b, respectively. The practitioner can alternately pull gently on each of the suture threads 193a, 193b, as indicated by arrows S to position the signal delivery device 130 at a precise location relative to the medial branch 180 (shown schematically in dotted lines in FIG. 4B). Allowing the practitioner to move the signal delivery device 130 by pulling (e.g., as opposed to pushing) is expected to improve the precision with which the practitioner can adjust the signal delivery device's position relative to the medial branch 180. The precise location can be identified by applying an electrical signal to the signal delivery device and observing the patient's motor response, as described above regarding the needle 191. The practitioner can use ultrasound and/or another suitable visualization technique, in addition to or in lieu of inducing a motor response. Any of these techniques can be performed iteratively until the electrodes are properly positioned. For example, in a representative process, the practitioner uses ultrasound to position the signal delivery device 130 close to the target location, and then iteratively applies the electrical signal while incrementally moving the signal delivery device and observing the patient's motor response until the target location is more precisely identified.

Referring now to FIG. 4C, the signal delivery device 130 has been positioned at the target location relative to the medial branch 180. The proximal suture thread 193a has been attached to the patient P at a proximal suture point 194a, and the distal suture thread 193b has been attached to the patient P at a distal suture point 194b. The suture points 194a, 194b can be shortened so as to not extend from the openings 195a, 195b; can be located subcutaneously but near the patient's skin, so as to be easily extracted if the need should arise; and/or can be elastic and/or otherwise configured to allow slight movement of the signal delivery device 130 once attached to the patient P. In particular embodiments, the sutures (e.g., the suture thread 193a, 193b, and/or other securing elements) can be made radiopaque or echogenic under fluoroscopy and/or ultrasound, so as to be more visible under fluoroscopy and/or ultrasound. For example, the suture can be secured with a fluoroscopic T-bar that is initially collapsed, and is then expanded into the adjacent tissue when in the proper position. Additionally, or alternatively, one or both of the suture threads 193a, 193b can be biodegradable. In these and other embodiments, the one or more anchors 137 (shown in FIG. 4A) can be deployed to secure the implantable device 120 in place.

FIG. 4D is another illustration of the implantable device 120, with the signal delivery device 130 positioned to direct an electrical field toward the medial branch 180 of the hypoglossal nerve HGN. As shown in FIG. 4C, the signal delivery device 130 can be positioned between the planes defined by the mylohyoid (which is out of the plane of FIG. 4D), the genioglossus and the hyoglossus muscles. Accordingly, in some embodiments, the signal delivery device 130 can abut or be close to the surface of the genioglossus muscle, without penetrating into the genioglossus muscle. In other embodiments, the signal delivery device 130 can penetrate into the genioglossus, which can aid in supporting the signal delivery device at its target location. The signal delivery device 130 can be positioned anterior to the anterior edge of the hyoglossus (as shown in FIG. 4D) so as to direct therapeutic signals to the medial branch 180, and/or can have other suitable positions, e.g. closer to the medial branch 180 (as shown in FIGS. 3C-3D). In some embodiments, the implantable device 120 can be positioned anteriorly relative to the position shown in FIG. 4D, such that the signal delivery device 130 can be positioned to direct an electrical field toward one or more branches 184a-b of the HGN and/or at or proximate to the motor end plate where the HGN and/or one or more of the branches 184a-b thereof innervate the tongue T. For example, in addition to or in lieu of positioning the implantable device 120 as shown, a first implantable device 120a (shown schematically) can be positioned such that a first signal delivery device 130a (shown schematically) is positioned to direct an electrical field toward a first branch 184a of the HGN, and/or a second implantable device 120b (shown schematically) can be positioned such that a second signal delivery device 130b (shown schematically) is positioned to direct an electrical field toward a second branch 184a of the HGN.

FIG. 4E is a coronal view taken through the patient's oral cavity, illustrating the implantable device 120 in a representative position. The device 120 is seen in cross-section as it extends into and out of the plane of FIG. 4E. In this position, the device 120 is just lateral from the hyoglossus and just medial from the mylohyoid at or near the point at which the planes of these two muscles cross. The device 120 is positioned just inferior to the HGN, which also extends into and out of the plane of FIG. 4E

An advantage of the foregoing approach is that the practitioner can move the signal delivery device 130 back and forth to find a precise target location, without having to make an incision in the patient. Instead, the signal delivery device is introduced into the patient percutaneously, which can improve patient outcomes, for example, by reducing the likelihood for an infection to develop. In addition, while anchors 137 (FIG. 4A) may be used to secure the signal delivery device 130 in position, in at least some embodiments, the suture threads 193a, 193b are sufficient to do so. In still further embodiments, the signal delivery device 130 can be held in position solely by the forces provided by the adjacent muscles, e.g., the mylohyoid, the genioglossus and the hyoglossus muscles, or by virtue of penetrating into the genioglossus, as discussed above, with suture threads and/or other anchor devices. Still further, in any of the foregoing embodiments, the signal generation function and the signal delivery function can be performed by initially separate elements, which are joined during the implant process, as discussed in further detail with reference to FIG. 6B.

Any of the techniques described herein for implanting the signal delivery device 130 can include one or more additional operations. For example, the practitioner can compress or otherwise manipulate (e.g., with his/her fingers) the submandibular or intraoral tissue to facilitate positioning the signal delivery device. These methods can allow the practitioner to manipulate the trajectory of the implant needle toward a desired endpoint. The additional force can be in form of manual pressure applied intra- or extraoral, and/or vacuum that is targeted to move tissue as a way of improving the precision with which the signal delivery device is implanted. Pressure and/or suction can also be used to avoid structures, such as glands.

The foregoing discussion with reference to FIGS. 3A-4E focused on electrodes positioned to deliver signals to the medial branch 180 of the hypoglossal nerve. As discussed previously, it is expected to be advantageous to apply electrical signals to the ansa cervicalis and/or directly to one or more of the muscles innervated by the ansa cervicalis, in addition to or in lieu of applying signals to the medial branch. FIG. 5A is a partially schematic illustration of the hypoglossal nerve and the ansa cervicalis, illustrating three branches of the ansa cervicalis that innervate the omohyoid, the sternothyroid, and the sternohyoid muscles. FIG. 5A also illustrates three representative signal delivery devices 130 (shown as devices 130a, 130b, 130c), each of which is positioned to direct electrical signals to a corresponding one of the branching nerves. In the illustrated embodiment, each signal delivery device 130 can include a lead body 134, carrying electrodes that are positioned to direct signals to the corresponding nerve, and a housing 135 that carries elements for receiving power from a remote power source, and generating the signals that are then supplied to the electrodes. In other embodiments, as described above regarding FIG. 4A, the lead body 134 can be omitted and the electrodes can be carried by the housing 135; this can reduce the overall size (e.g., length) of the implantable device and improve the practitioner's ability to precisely position the signal delivery device 130 at or proximate the target nerve. The approach discussed above with reference to FIGS. 4A-4C, can also be used to position the signal delivery device 130 proximate to the ansa cervicalis. Further details of representative signal delivery devices 130 suitable for any of the foregoing locations are described below with reference to FIGS. 6A-6C.

In some embodiments, electrical signals can be applied to multiple different targets. For example, FIG. 5B illustrates two signal delivery devices 230 (individually identified as a first signal delivery device 230a and a second signal delivery device 230b, described in greater detail with reference to FIG. 6C). The first signal delivery device 230a is positioned to deliver a first electrical signal (schematically represented as a first electrical field E1) to the medial branch 180 of the hypoglossal nerve HGN and the second signal delivery device 230b is positioned to deliver a second electrical signal (schematically represented as a second electrical field E2) to the ansa cervicalis nerve AC. In other embodiments, the first signal delivery device 230a and/or the second signal delivery device 230b can be positioned to directly stimulate one or more of the muscles at or near their respective locations. For example, the first signal delivery device 130a can be positioned to deliver the first electrical signal/field E1 to the hyoglossus muscle and/or the genioglossus muscle, and/or the second signal delivery device 130b can be positioned to deliver the second electrical signal/field E2 to the thyrohyoid muscle, the sternohyoid muscle, the omohyoid muscle, and/or the sternothyroid muscle. Although the signal delivery devices 230 illustrated in FIG. 5B are leadless, in other embodiments leaded signal delivery devices, such as the signal delivery device 130, can be positioned as shown in FIG. 5B.

### 5. Representative Signal Delivery Devices

FIG. 6A is a partially schematic side view of an implantable device 120 having elements configured in accordance with representative embodiments of the present technology. In an embodiment shown in FIG. 6A, a single implantable device 120 performs both signal generation functions and signal delivery functions. Accordingly, the implantable device 120 includes both the implantable signal delivery device 130, and an implantable signal generator 110. Representative dimensions are indicated in FIG. 6A to provide a sense of scale, but the technology is not limited by these dimensions unless expressly stated. The signal delivery device 130 includes a lead body 134, which can be generally flexible, and can carry one or more electrodes 131, which are generally rigid in some embodiments, and may be flexible in others. Flexible electrodes can increase the flexibility of the lead body generally to accommodate the tortuous anatomy/insertion path near the target nerve. For purposes of illustration, the lead body 134 is shown as carrying four electrodes 131 in FIG. 6A, but in other embodiments, the lead body 134 can carry other suitable numbers of electrodes, for example, two electrodes 131. The electrodes 131 can be arranged in an array, for example, a one-dimensional linear array. The electrodes 131 can include conventional ring-shaped, or cylindrical electrodes, manufactured from a suitable, bio-compatible material, such as platinum/iridium, stainless steel, MP35N and/or or other suitable conductive implant materials. The electrodes 131 can each be connected to an individual conductor 140, for example, a thin wire filament, that extends through the lead body 134. Each electrode 131 can have a length of approximately 1.5 mm as shown in FIG. 6A, or another suitable length in other embodiments. In particular embodiments, portions of the electrode(s) may be directional, circumferentially masked, and/or segmented to more precisely target the electrical field in a clockwise or counterclockwise direction around the longitudinal axis of the lead body 134. This technique can be used to direct the electrical field away from the retrusers (as discussed above with reference to FIGS. 3C and 3D), and/or to avoid the alveolar nerve and/or other sensory nerves in the vicinity. This approach includes rotating the electrodes 131 (or maintaining the rotational position of the electrodes 131) to have the proper clock position relative to the target neural population.

In the embodiment illustrated in FIG. 6A, the lead body 134 is connected to, and carried by, a housing 135, which in turn carries the signal generator 110 and circuit elements for receiving power. For example, the overall housing 135 can include an antenna housing or housing portion 135a and a circuit housing or housing portion 135b. The antenna housing 135a may be flexible, and can carry a receiver antenna 133 (or other suitable power reception device), which receives power from the wearable device 101 (FIG. 2) via the wireless transmission link 114. The circuit housing 135b can have the form of a generally cylindrical metallic “can” formed from titanium, platinum, a platinum-iridium alloy, a ceramic, and/or another suitable material and/or combination thereof. The signal generator 110 can include a charge pump and/or DC-DC converter 139 and/or circuitry 138 (e.g., second circuitry) coupled to the receiver antenna 133. In some embodiments, the electrode receiver antenna 133 can be coupled to an AC-DC convertor configured to convert the received power signal (e.g., via the RF transmission link 114, shown in FIG. 2) to DC current. The circuitry 138 can include an ASIC, which can in turn include corresponding machine-readable instructions. The instructions can be updated wirelessly, using the electrode receiver antenna 133 for data transfer in addition to power transfer. For example, data can be transferred using pulse-width modulation (PWM) and/or other suitable techniques. Data can also be transferred in the opposite direction, e.g., using backscatter and/or other suitable techniques. For example, the implantable device 120 can transmit a receipt to indicate that power has been received, and what magnitude the power is. This information can be used to autoregulate (up or down) the output of the signal generator 110, e.g., the transmitted signal and phase. Accordingly, the circuitry 138 can include a processor and memory, including pre-programmed and updatable instructions (e.g., in the form of an ASIC) for delivering therapy signals to the patient. For example, the system can include boot loader embedded firmware. Furthermore, the overall system can use RFID-type power transmission authorization to discriminate between multiple implantable devices, which may be powered by a single wearable device 101. RFID and/or other techniques can be used to implement security measures, e.g., to ensure that no foreign or unintended stimulation occurs. Such techniques can be implemented with suitable hardware/software carried by the implantable device 120, in at least some embodiments.

The overall housing 135 can further include a base 136, which is generally rigid, and one or more anchors 137. The anchor(s) 137 can be used in addition to or in lieu of the suture threads shown in FIG. 4C to securely position the implantable device 120 relative to the patient's tissue. In a representative embodiment, the anchor 137 includes one or more tines that extend outwardly and into the patient's tissue when the implantable device 120 is injected or otherwise implanted in the patient. In other embodiments, the implantable device 120 can include other suitable anchors, and/or anchoring may occur at the distal and/or mid-section of the signal delivery device 130. Other suitable anchors include but are not limited to: (a) a bow spring that runs the longitudinal length of the electrode array and bows out to create fixation friction when the introducer sheath is withdrawn; (b) a small wire on a spring-loaded hinge that runs the longitudinal length of the electrodes array and bows out to create fixation friction when the introducer sheath is withdrawn; (c) a cam that, when rotated, expands in diameter to create frictional fixation when the corresponding push rod is rotated by the implanter; and/or (d) a torsion spring that, when rotated, expands in diameter to create frictional fixation when the push rod is rotated by the implanter.

Other suitable anchoring techniques include bending or deforming the lead body 134 so that it is biased into contact with the walls of the channel formed by the insertion needle. The lead body can have a bend that is straightened out during insertion (e.g., via a stylet, or by virtue of being constrained within introducer or cannula), but which re-forms and produces an anchoring force when the constraint is removed. In still a further technique, the distal end of the lead body 134 is buckled (in an axial or columnar direction) once at the target location. The buckling action locally expands the diameter of the lead body so as to expand it against the tissue in which it is placed. For instances in which the device is implanted temporarily, the stylet used to introduce the device can include a bend or kink.

Yet further techniques for securing the lead body and/or other implantable element include using a mesh. For example, a plug or mesh can be inserted of over at least a portion of an already deployed lead body to improve anchoring. Accordingly, the plug or mesh is not integral with the lead body 134 when the lead body is injected, but is instead added to secure the lead body after the lead body is in place. The plug or mesh can be expanded radially in the manner of a suture sleeve to secure the lead body 134 against the adjacent tissue. The plug or mesh can be applied as a temporary anchor or it can for the basis for a chronic anchor. Like the other elements described above, the plug or mesh can be delivered via injection.

In at least some instances, the plug or mesh described above can have acute as well as (or in lieu of) long term or chronic applications. For example, if the practitioner induces a hemorrhage or a subsequent infection occurs, the plug/mesh can be used to manage or minimize negative sequalae, e.g., by stopping a hemorrhage.

In operation, the receiver antenna 133 receives power wirelessly from the power source 109 carried by the associated wearable device 101 (FIG. 2). In at least some embodiments, the power received at the receiver antenna 133 is in a range, for example, a radio frequency in a range of from about 400 MHz to about 2.5 GHz, e.g., from about 600 MHz to about 2.45 GHz, between about 900 MHz to about 1.2 GHz, or any other frequency or frequency range therebetween. At this frequency, the useable range of the wireless power transmission link 114 is about 10 cm, more than enough to cover the distance between the implanted signal delivery device 130 and the wearable device 101. At this range, the power transmission process is not expected to cause tissue heating, and accordingly provides an advantage over other power transmission techniques, for example, inductive transmission techniques. However, in embodiments for which the potential heating caused by inductive power transmission is adequately controlled, inductive techniques can be used in lieu of the midfield power transmission techniques described herein.

The AC power received at the receiver antenna 133 is rectified to DC (via, e.g., an AC-DC converter), then transmitted to a DC-DC converter, charge pump, and/or transformer 139, and converted to pulses in a range from about 10 Hz to about 500 Hz, such as from about 30 Hz to about 300 Hz. In other embodiments, the pulses can be delivered at a higher frequency (e.g., 10 kHz or more), and/or in the form of bursts. The amplitude of the signal can be from about 1 mV to about 5V (and in particular embodiments, 1 V to 2 V) in a voltage-controlled system, or from about 0.5 mA to about 12 mA in a current-controlled system. The circuitry 138 controls these signal delivery parameters, and transmits the resulting electrical signal to the electrodes 131 via the wire filaments or other conductors 140 within the lead body 134. Accordingly, the circuitry forms (at least part of) the signal generator 110 in that it receives power that is wirelessly transmitted to the implantable device 120, and generates the signal that is ultimately delivered to the patient. The electrical field(s) resulting from the currents transmitted by the electrodes 131 produces the desired effect (e.g., excitation and/or inhibition) at the target nerve. In at least some embodiments, the implantable device 120 need not include any on-board power storage elements (e.g., power capacitors and/or batteries), or any power storage elements having a storage capacity greater than 0.5 seconds, so as to reduce system volume. In other embodiments, the implantable device 120 can include one or more small charge storage devices (e.g., capacitors, solid state batteries, and/or the like) that are compatible with the overall compact shape of the implantable device 120, and have a total charge storage capacity of no more than 1 second, 30 seconds, 1 minute, 2 minutes, or 5 minutes, depending on the embodiment.

FIG. 6B is a partially schematic illustration of an implantable device 120 configured in accordance with further embodiments of the present technology. One feature of this embodiment is that the overall housing 135 (carrying the signal generator 110) and the lead body 134 are initially separate elements. Accordingly, the lead body 134 can be introduced into the patient, then positioned at or near the target neural population, and then connected to the overall housing 135. One advantage of this approach is that the practitioner can select from among different lead bodies 134 having different lengths, choosing the lead body 134 having the appropriate length (and/or other configuration attribute) for the particular patient undergoing therapy. Another advantage is that the diameter of the tunnel into which the (small diameter) lead body 134 is positioned can remain small enough to accommodate only the lead body 134, and not the (larger diameter) overall housing 135. This approach can reduce trauma to the tissue and allow the patient to achieve a therapeutic endpoint. Other techniques can also be used to further the foregoing results. For example, the tunnels (or at least portions of the tunnels) into which the signal generator 110 and/or the signal delivery device 130 fit, can be formed via tissue dilation rather than cutting. In addition to being less traumatic, this approach can produce tissue compression around the signal generator 110 and/or the signal delivery device 130, which can at least reduce the tendency for these elements to migrate.

The overall housing 135 can be positioned at, or very close to, an entry opening into the patient's tissue. This approach has the added advantage that the overall housing 135, which includes the receiver antenna 133, will be positioned close to the patient's skin, which reduces power losses associated with transmitting power through the patient's skin to the signal delivery device 130. Because power losses typically produce heat, this approach can also reduce tissue heating.

The lead body 134 can include multiple electrodes 131 positioned toward its distal end. For purposes of illustration, four electrodes 131 are shown in FIG. 6B, but in other embodiments, the signal delivery device 130 can include other numbers of electrodes 131. Each electrode 131 is coupled to a corresponding first terminal 129a via a corresponding conductor 140 (not visible in FIG. 6B). The lead body 134 can have an overall length L that has any of a number of suitable predetermined/standard (or non-standard) values. The lead body 134 can include an axial lead opening 128a, for example, if the lead body 134 is delivered into the patient via a stylet. The stylet is then removed before connecting the lead body 134 to the overall housing 135. In other embodiments, no stylet is required, and instead, the lead body 134 is housed in the lumen of a needle, introducer, or sheath, and then deployed into the patient as the needle, introducer, or sheath is withdrawn from the patient.

The overall housing 135 includes an antenna housing 135a and circuit housing 135b at least generally similar to those discussed above with reference to FIG. 6A. The overall housing 135 can further include a connector housing 135c that houses second terminals 129b, shaped and positioned to receive the first terminals 129a of the lead body 134. The connector housing 135c can be partly or completely flexible. The second terminals 129b can be partly rigid, with flexible components (e.g., springs) to provide resilient physical and electrical contact with the first terminals 129a. In particular embodiments, the second terminals 129b can include donut-shaped terminals positioned along an axial housing opening 128b. Representative second terminals are manufactured by Bal Seal Engineering, Inc. of Lake Forest, Calif. In operation, the practitioner introduces the lead body 134 into the patient separately from the overall housing 135, for example, via a stylet. The lead body 134 is then connected to the overall housing 135 by inserting the lead body 134 into the housing axial opening 128b as indicated by arrow B. If the lead body 134 has previously been secured in position, then all or most of the insertion motion is undertaken by the overall housing 135, not the lead body 134. The overall housing 135 can be secured in position via one or more anchors 137, and/or sutures. If, in the unlikely event that either the lead body 134 or the overall housing 135 need to be replaced, each can be replaced separately from the other by separating the lead body 134 from the overall housing 135.

Because the lead body 134 and portions of the overall housing 135 are flexible, in addition to being separable, each of these components can have a different orientation when inserted into the patient's tissue. For example, the lead body 134 can extend at a shallow or steep angle into the patient's tissue to access the target nerve. The overall housing can extend at a shallower angle (e.g., parallel to the patient's skin surface) to position the antenna 133 for better (e.g., optimal) power reception). However, both elements can be introduced into the patient through the same opening, thus limiting the invasiveness of the implant procedure. In addition, the proximity of the overall housing 135 to the opening reduces the length of the sheath and/or other introducer required to position the overall housing 135 at its target location. In other embodiments, the lead body 134 can be delivered using both a distal and proximal opening, as discussed above with reference to FIG. 4A-4C, and the overall housing can be delivered via only the distal opening 195.

Whether the implantable device 120 is implanted as a single unit or as two initially separated units, the technique of placing different portions of the implantable device 120 into tunnels have different diameters (as described above), can apply. This approach can more firmly secure elements of the implantable device 120 in place. For example, the implantation process can include inserting a small diameter guide wire (e.g., 0.014″), without further dilation, to form the distal 5-30 mm of the tunnel. This portion of the tunnel can snuggly accommodate the (small diameter) lead body 134. The portion of the tunnel that snuggly accommodates the (larger diameter) overall housing 135 can have a slightly larger diameter, e.g., 7 Fr (2.33 mm) to 8 Fr (2.66) mm. In the foregoing example, the lead body 134 can have a diameter of 3 Fr (1 mm), and the overall housing 135 can have a diameter of 6 Fr (2 mm). In other embodiments, these diameters can be different (larger or smaller) and the tunnel diameters adjusted accordingly. This approach can eliminate the need for tines or other slightly more invasive anchors. As described above, the opening(s) that accommodate the implantable device 120 can be formed primarily via dilation/dilatation, to reduce tissue trauma and/or improve device anchoring.

In at least some embodiments, the electrical signal delivered to the patient can be delivered via a bipole formed by two of the electrodes 131. In other embodiments, the signal can be a monopolar signal, with the housing 135 (e.g., the circuit housing 135b) forming a ground or return electrode. In general, the waveform includes a biphasic, charge balanced waveform, as will be discussed in greater detail below with reference to FIGS. 7A and 7B.

FIG. 6C is a side view of a further representative implantable device 220 including a leadless signal delivery device 230 configured in accordance with embodiments of the present technology. At least some aspects of the leadless signal delivery device 230 can be generally similar or identical in structure and/or function to the signal delivery device 130 of FIGS. 6A and/or 6B. Accordingly, like names and/or reference numbers (e.g., housing 235 of FIG. 6C versus the housing 135 of FIG. 6A) are used to indicate generally similar or identical components. The leadless signal delivery device 230 includes a housing 235 having a first housing portion 235a, a second housing portion 235b, and a base 136. The first housing portion 235a can be generally similar to the antenna housing 135a, and/or can have a first outer dimension D1 (e.g., a first width, a first diameter, a first circumference, and/or the like). The second housing portion 235b can be generally similar to the circuity housing 135b, and/or can have a second outer dimension D2 (e.g., a second width, a second diameter, a second circumference). In the illustrated embodiment the first outer dimension D1 is less than the second outer dimension D2. In other embodiments, the first outer dimension D1 can be equal to or greater than the second outer dimension D2. The base 136 can include one or more of the anchors 137.

The leadless signal delivery device 230 can further include the electrode receiver antenna 133, the signal generator 110, the circuitry 138, the charge pump 139, and the one or more electrodes 131. In the illustrated embodiment, the electrode receiver antenna 133 is positioned within the first housing portion 235a, the signal generator 110, the circuit 138, and the charge pump 139 are positioned within the second housing portion 235b, and the electrodes 131 are carried by the second housing portion 235b. For example, as shown in FIG. 6C, the electrodes 131 are positioned to be exposed from an exterior surface of the second housing portion 235b, such that individual ones of the electrodes 131 extend at least partially around a circumference of the second housing portion 235b. Accordingly, one or more of the electrodes 131 can extend at least partially or fully around (e.g., circumferentially around, axially around, etc.,) one or more of the internal components of the leadless signal delivery device 230. In the illustrated embodiment, the signal generator 110, the circuitry 138, and the charge pump 139 are each positioned within the second housing portion 235b such that one or more of the electrodes 131 extend at least partially around each of the signal generator 110, the circuitry 138, and the charge pump 139. More specifically, in the illustrated embodiment the electrodes 131 and/or the second housing portion 235b define an axial space or volume within which each of the signal generator 110, the circuitry 138, and the charge pump 139 are positioned. Additionally, or alternatively, the electrode receiver antenna 133 can be positioned within the second housing portion 235b, such that one or more of the electrodes 131 can extend at least partially around the electrode receiver antenna 133. In such embodiments, the second housing portion 235b can be configured to reduce or prevent interference with the electrode receiver antenna's reception of the power transmission link 114. The electrodes 131 and/or the second housing portion 235b are not expected to interfere with the operation of the electrode receiver antenna 133. Additionally, or alternatively, one or more electrodes can be positioned on or at the first housing portion 235a to extend at least partially around the electrode receiver antenna 133. In these and other embodiments, one or more of the signal generator 110, the circuitry 138, and/or the charge pump 139 can be positioned within the first housing portion 235a and/or otherwise positioned outside and/or laterally relative to the space defined by the electrodes 131 and/or the second housing portion 235b.

Each of the electrodes 131 can be coupled to the signal generator 110 via a respective conductor 140. In the illustrated embodiment, each of the conductors 140 are positioned within the second housing portion 235b, for example, between the signal generator 140 and an inner surface of the second housing portion 235b. Additionally, or alternatively, one or more feedthroughs 143 can couple individual ones of the conductors 140 to the signal generator 110.

### 6. Representative Waveforms

The signal generators and delivery devices described above can generate and deliver any of a variety of suitable electrical stimulation waveforms to modulate the actions of the patient's neurons and/or muscles. Representative examples are illustrated in FIGS. 7A and 7B and include a series of a biphasic stimulation pulses that form stimulation wave cycles having a period as identified in FIGS. 7A and 7B. The waveform parameters can include active cycles and rest cycles. Each period P includes one or more pulses. The waveform shown in FIG. 7A comprises an anodic pulse followed by an interphasic delay, a cathodic pulse and then an interpulse delay. Accordingly, the overall period P or cycle includes the following parameters: anodic pulse width (PW1), anodic amplitude (e.g., voltage or current amplitude VA), interphasic delay/dead time, cathodic pulse width (PW2), cathodic amplitude (e.g., voltage or current amplitude VC), interpulse delay/idle time, and peak-to-peak amplitude (PP). The parameters may also include the identity of the electrode(s) to which the signal is directed. The anodic pulse width (PW1) in some representative embodiments is between 30 μs and 300 μs. The anodic amplitude (VA) in some representative embodiments ranges from 1 mV to 5 V, or 1 mA to 10 mA. The interphasic delay in some representative embodiments can be from 10 μs to 100 μs. The cathodic pulse width (PW1) is some representative embodiments is between 30 μs and 300 μs. The cathodic amplitude (VA) in some representative embodiments ranges from 0.3V to 5V. In representative embodiments, the anodic and cathodic phases are charge balanced, though the phases need not be symmetrically shaped. The interpulse delay in some representative embodiments can be from 10 μs to 250 μs. The peak-to-peak amplitude in some representative embodiments can be from about 2 mA to 12 mA. Representative frequencies range from about 10 Hz to about 500 Hz, such as from about 30 Hz to about 300 Hz in some embodiments, and up to 100 kHz (e.g., 10 kHz) in others. The pulses can be delivered continuously or in bursts. The frequency, the frequency range, the amplitude (e.g., peak-to-peak amplitude), the interpulse delay, the pulse width, and/or other signal delivery parameters can be varied based at least partially on the implanted location and/or the stimulation target of the signal delivery device 130. In some embodiments, multiple signal delivery devices are implanted in a patient, and each signal delivery device is configured to deliver a respective electrical signal having one or more respective signal delivery parameters. For example, a first signal delivery device implanted at a first location can be configured to deliver a first electrical signal having one or more first signal delivery parameters, a second signal delivery device implanted a second location can be configured to deliver a second electrical signal having one or more second delivery parameters, and individual ones of the first signal delivery parameters (e.g., amplitude, frequency, etc.) can be the same and/or different than individual ones of the second signal delivery parameters. Continuing with this example, the first electrical signal can include a first frequency and/or a first amplitude, the second electrical signal can include a second frequency and/or a second amplitude, and the first frequency can be the same or different than the second frequency and/or the first amplitude can be the same or different than the second amplitude.

FIG. 7B illustrates a representative waveform comprising an active portion and a rest portion. The active portion includes one or more periods having the characteristics described above with reference to FIG. 7A. The rest portion has no stimulation pulses. According to some representative embodiments, the ratio of active portion to rest portion can be between 1:1 and 1:9. As a representative example, if the ratio is 1:9, and there are 300 active periods, there can be 2700 rest portions.

In a representative example, the stimulation voltage may be presented independently to each contact or electrode. For the positive pulse, the positive contact can be pulled to the drive voltage and the negative contact is pulled to ground. For the negative pulse, the negative contact can be pulled to the drive voltage and the positive contact is pulled to ground. For dead time and idle time, both contacts are driven to ground. For the rest time, both contacts are at a high impedance. To prevent DC current in the contacts, each half-bridge can be coupled to the contact through a capacitor, for example, a 100 μF capacitor. In addition, a resistor can be placed in series with each capacitor to limit the current in the case of a shorted contact. The pulses of the therapeutic waveform cycle may or may not be symmetric, but are generally shaped to provide a net-zero charge across the contacts.

### 7. Further Implant Techniques

FIGS. 4A-4C and the associated discussion described techniques for implanting an electrode using a curved needle with both an entry and an exit point in the patient's skin. The following representative implantation technique is performed with a single puncture.

### 7.1 Procedure

### 7.1.1 Materials

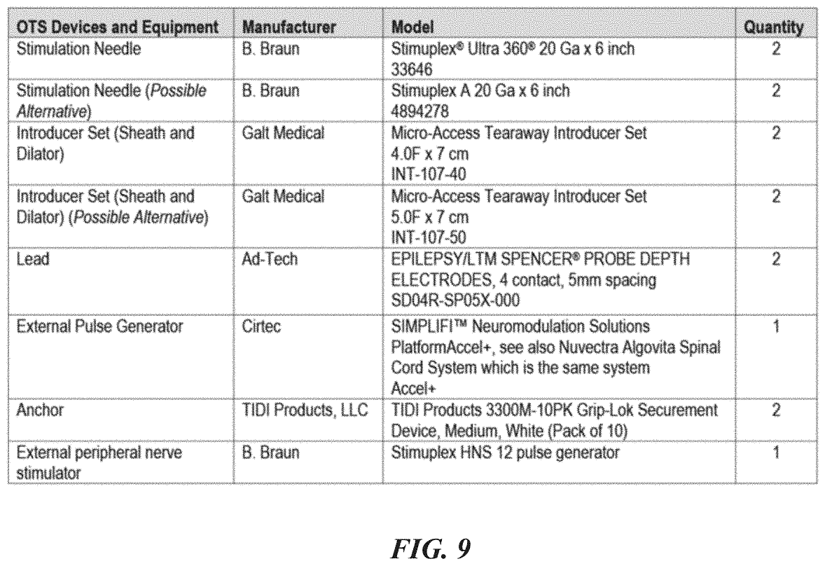

FIG. 8 outlines the overall procedure. Representative materials are listed below and in FIG. 9.
Basic surgical instruments (i.e., forceps, scalpels, etc.).
Ultrasound system with color Doppler capabilities, 12L ultrasound probe, and ultrasound gel.

### 7.1.2 Preparation for the Procedure

Flush dilator and/or sheath with sterile saline.
Thread stimulating needle through dilator.
Thread needle and dilator through split sheath. Flush the needle with sterile saline.

### 7.1.3 Preparation for the Patient

Place the patient in the supine position with head supported by a foam ring and the surgeon above the head of the bed. Ask patient to rotate head to the left or right, extending the neck comfortably.

### 7.1.4 Hypoglossal Nerve Localization and Identification of Relevant Anatomy

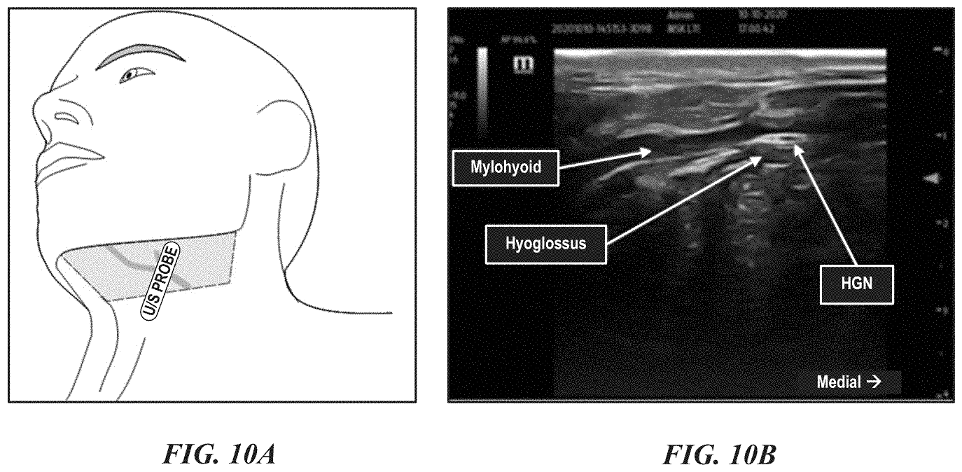

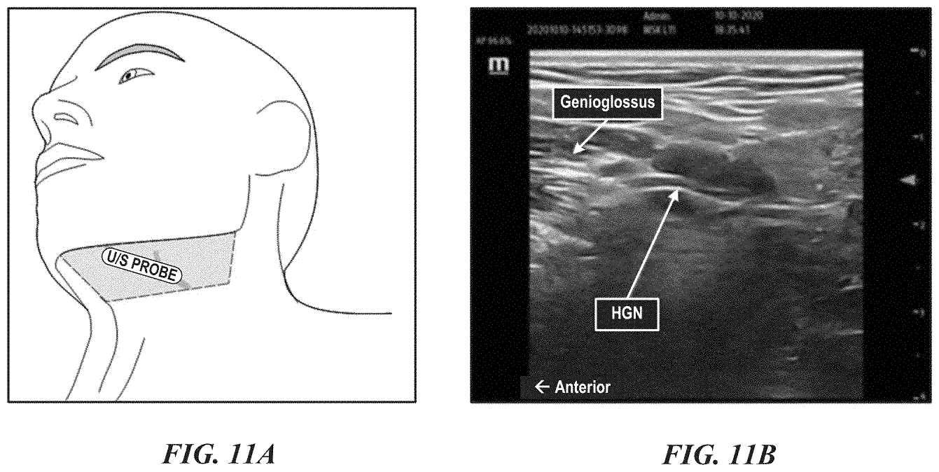

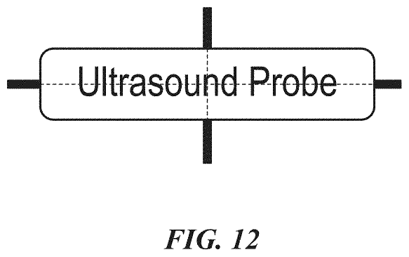

Placing the ultrasound probe to lie between the hyoid bone and the approximate midpoint of the edge of the mandible, identify the hypoglossal nerve in the coronal view between the mylohyoid and hyoglossus muscles (FIGS. 10A, 10B).
While constantly maintaining the view of the HGN, rotate the probe to image a parasagittal view of the nerve with the longest visible length. Identify the leading edge of the hyoglossus muscle and the most distal portion of the nerve prior to diving into the genioglossus muscle (FIGS. 11A, 11B).
Using color Doppler ultrasound, identify vasculature in the area.
Identify submandibular and sublingual salivary glands in ultrasound imaging.
Identify the optimal submandibular and/or intraoral insertion point that will allow for delivery system and electrode to be placed as close to parallel to the nerve as possible.
   External needle guide may be used to better align the needle insertion point to the ultrasound image.
   Investigate if pushing or pulling the submandibular or intraoral tissues would improve the parallel alignment between the implant tool/lead pathway and HGN. If a such configuration exists, apply the necessary tissue manipulation with available tools.
Using a skin marker, mark the position of the probe by marking the ends and the center of the probe (FIG. 12).

### 7.1.5 Administration of Anesthesia

Administer Conscious Sedation, General Anesthesia, and/or Local Anesthesia as indicated by the clinician and consented to by the patient.

### 7.1.6 Electrode Insertion

### Localization of Target Electrode Position

Holding the dilator and sheath as proximally as possible (usually at the hub), insert stimulating needle using ultrasound guidance to align the trajectory of the needle as close to the HGN as possible. The leading edge of the hyoglossus muscle and the most distal portion of the hypoglossal nerve that is visible may be used as the most proximal and distal references for the needle trajectory. To achieve an angle as parallel as possible along the nerve, needle may be inserted normal to the patient's skin/tissue(s) or at an exaggerated angle then tilted to the desired angle, as discussed above with reference to FIG. 4A.
More generally, applying stimulation prior to implanting the implantable device can be an important navigation method for identifying the right location to elicit the desired response, and therefore locate a chronic implant. Because the smaller stimulation needle provides good ultrasound contrast, it can operate as a “navigation waypoint,” creating a path to follow when implanting the signal delivery device. This technique can be used to deliver multiple signal delivery devices from either a single entry point, or multiple entry points. Multiple signal delivery devices can provide additional assurance that a suitable therapeutic location or locations will be identified.
If inserting the needle posterior to the target area of the nerve, the insertion point should be aligned with the center plane of the ultrasound probe and 5-30 mm posterior to the posterior end of the ultrasound probe.
If inserting the needle anterior to the target area of the nerve, the insertion point should be aligned with the center plane of the ultrasound probe and posterior to the inner edge of the mandible.
Observe insertion procedure and check for excessive blood flow.
Connect the stimulating needle to the peripheral nerve stimulator. Use a sterile cover if necessary.
Apply electrical stimulation using the stimulating needle. Representative parameters include: a frequency in a frequency range from 1 to 50 Hz, such as 40 Hz, 1 to 3 Hz, or 1 to 2 Hz; an amplitude between 0.25 to 5 volts, such as 1.5 volts, or 0.5 to 5 mA; a pulse width between 25 to 250 μs, such as 150 μs.
Slowly increase stimulation amplitude, looking for protrusion of the tongue (i.e., genioglossus activation) and minimal retrusion or dipping of the oral tongue inferiorly (styloglossus and hyoglossus activation).
If no response or undesired response is seen, turn off stimulation and adjust the needle slightly under ultrasound guidance.
Once an appropriate stimulation response is achieved, disconnect the needle from the peripheral nerve stimulator.

### 7.1.7 Sheath Delivery

Holding needle hub in place, advance the dilator over the stimulating needle under ultrasound guidance until distal end of dilator meets the tip of the needle.
Holding the needle hub and dilator in place, advance the sheath under ultrasound guidance until distal end of sheath meets the tip of the needle and dilator.
Withdraw the needle and the dilator while leaving the sheath in position.

### 7.1.8 Implantable Electrode Array Placement

Insert implantable electrode array (e.g., implantable device 120, signal delivery device 130, a linear array of electrodes carried by a lead body, and the like) through sheath under ultrasound guidance until it is visualized protruding through the end of the sheath.
Remove sheath while holding the electrode array in place.
If possible, confirm under ultrasound that the electrode array has not migrated. NOTE: It may not be possible to view the hypoglossal nerve under the shadow cast by the array.
Secure implantable electrode lead body with external anchor provided on the surface of the skin at the entry location, allowing some slack for movement due to tongue response, and/or internal anchor carried by implantable electrode lead body.
Internal anchors can include a deformed lead or stylet, plugs, tines, mesh, springs, suture ends, helices, etc. and may be used to improve stability.
Connect the power source to the implantable electrode array. This can include aligning a power transmission antenna operably coupled to the power source with an electrode receiver antenna operably coupled to the implantable electrode array by, for example, placing the power supply above/proximate to the implantable electrode array. Use a sterile cover if necessary.

### 7.1.9 Stimulation Protocol

Using sterile technique, bag the power source.
Confirm that the power source is set to minimum amplitude/frequency. Supply power to implantable electrode array and begin stimulation.
Increase stimulation amplitude and/or frequency until a physiological response of the stimulation is observed.
Check for physiological responses to the stimulation including:
   Tongue protrusion
   Tongue retrusion
   Inferior dipping of oral tongue
   Flow measurement(s), such as air flow through the patient's airway.
   Other observed physiologic responses
Deactivate stimulation.
Repeat the Stimulation Protocol for other electrode configurations if required.
If desired response is not detected, the external or internal anchors may be loosened/retracted, the lead may be incrementally retracted, resecured, and retested.
Set final stimulation amplitude
Close wound with suture
Recover Patient

From the foregoing, it will be appreciated that specific embodiments of the disclosed technology have been described herein for purposes of illustration, but that various modifications may be made without deviating from the technology. For example, the power source and associated wearable can have configurations other than an intraoral mouthpiece, that also deliver power wirelessly to one or more implanted electrodes. Representative configurations include external, skin-mounted devices, and devices that are worn around the patient's neck, which may be suitable for targeting the ansa cervicalis, vagal nerve, and/or other nerves other than the HGN. Other representative targets for the stimulation include palatoglossal stimulation, cranial nerve stimulation, direct palatoglossus muscle stimulation, hyolaryngeal stimulation, and/or glossopharyngeal nerve stimulation. The anchor used to secure the signal delivery device in place can have configurations other than deployable tines, including s-curve elements, helixes, and/or porous structures that promote tissue in-growth. Or, as was discussed above, the anchors can be eliminated and replaced with sutures. The signal delivery device was described above as including multiple housings that form an overall housing. In other embodiments, the multiple housing can be portions of a unitary overall housing.

Certain aspects of the technology described in the context of particular embodiments may be combined or eliminated in other embodiments. For example, signal delivery devices having any of a variety of suitable configurations can be used with any one signal generator, and signal generators having any of a variety of suitable configurations can be used with any one signal delivery device. Further, while advantages associated with certain embodiments of the disclosed technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

As used herein, the phrase “and/or,” as in “A” and/or “B” refers to A alone, B alone and both A and B. To the extent any materials incorporated herein by reference conflict with the present disclosure, the present disclosure controls.

To the extent any materials incorporated herein by reference conflict with the present disclosure, the present disclosure controls.

The following examples provide additional representative features of the present technology.

### EXAMPLES

1. A method for treating a patient, comprising:
percutaneously implanting a signal delivery device at a target signal delivery location in a patient, wherein the signal delivery device includes an electrode, and wherein the electrode is positioned to produce a net positive protrusive motor response of the patient's tongue; and
providing power to the electrode from a wearable power source to cause the electrode to deliver an electrical signal to the target signal delivery location to produce the net positive protrusive motor response.

2. The method of example 1 wherein percutaneously implanting the signal delivery device includes percutaneously implanting the signal delivery device alongside a medial branch of a patient's hypoglossal nerve, wherein the signal delivery device includes an electrode, and wherein the electrode is positioned inferior to the medial branch and inferior to at least one retruser extending from the medial branch.

3. The method of example 1 or example 2 wherein percutaneously implanting the signal delivery device includes percutaneously implanting the signal delivery device alongside a medial branch of a hypoglossal nerve of the patient, wherein the medial branch includes a retruser extending away from the medial branch in a first area, and wherein the signal delivery device includes an electrode positioned to deliver electrical stimulation to a second area opposite from the first area.

4. The method of any of examples 1-3 wherein the net positive protrusive response includes a retrusive response and a protrusive response greater than the retrusive response.

5. The method of any of examples 1-4 wherein providing the power to the electrode includes causing the electrode to deliver the electrical signal without activating any retruser of the patient.

6. The method of any of examples 1-5 wherein inducing the net positive protrusive motor response of the patient's tongue includes at least one of causing the patient's tongue to move anteriorly away from the patient's airway or inducing caudal traction of the patient's hyoid bone and/or thyroid cartilage.

7. The method of any of examples 1-6 wherein the target signal delivery location includes a hypoglossal nerve, an ansa cervicalis nerve, a genioglossus muscle, a geniohyoid muscle, a sternohyoid muscle, a thyrohyoid muscle, an omohyoid muscle, and/or a sternothyroid muscle of the patient.

8. The method of any of examples 1-7 wherein inducing the net positive protrusive motor response in the patient's tongue includes causing an airway of the patient to open or further open in response to delivery of the electrical signal.

9. The method of any of examples 1-8 wherein the signal delivery device is a first signal delivery device and the target signal delivery location is a first target signal delivery location, the method further comprising percutaneously implanting a second signal delivery device proximate a second target signal delivery location.

10. The method of any of examples 1-9 wherein providing power includes transmitting power to the electrodes via an RF link.

11. The method of example 10 wherein transmitting the power via the RF link includes transmitting the power at a frequency in a frequency range between 400 MHz and 2.5 GHz.

12. The method of example 10 or example 11 wherein transmitting the power includes transmitting the power at a frequency in a frequency range between 900 MHz and 1.2 GHz.

13. The method of any of examples 1-12 wherein providing the power to the electrode includes causing the electrode to deliver an electrical signal having at least one of:
an interpulse delay between 10 μs and 250 μs;
a peak-to-peak amplitude between 0.5 mA and 12 mA; or
a frequency in a frequency range between 10 Hz and 500 Hz.

14. The method of any of examples 1-13 wherein the electrode a plurality of circumferential segments and wherein providing the power to the electrode includes causing the electrode to deliver the electrical signal via individual ones of the circumferential segments of the electrode.

15. The method of any of examples 1-14, further comprising:
before implanting the signal delivery device, percutaneously inserting a needle into the patient along a trajectory toward a medial branch of the patient's hypoglossal nerve; and
aligning the needle with the medial branch, wherein implanting the signal delivery device includes directing the signal delivery device along the trajectory of the needle.

16. The method of example 15 wherein the electrical signal is a first electrical signal, the method further comprising delivering a second electrical signal to the patient via the needle to aid in positioning the signal delivery device.

17. The method of example 16 wherein the first electrical signal has a first signal delivery parameter, and wherein the second electrical signal has a second signal delivery parameter different than the first signal delivery parameter.

18. The method of any of examples 1-17 wherein percutaneously implanting the signal delivery device includes directing a percutaneous insertion device into the patient at a first location.

19. The method of example 18 wherein directing the percutaneous insertion device into the patient at the first location includes directing the percutaneous insertion device into the patient at a submandibular location.

20. The method of example 18 wherein directing the percutaneous insertion device into the patient at the first location includes directing the percutaneous insertion device into the patient at an intraoral location.

21. The method of example 20 wherein directing the percutaneous insertion device into the patient at the intraoral location includes directing the percutaneous insertion device into the patient at a sublingual location.

22. The method of any of examples 18-21 wherein percutaneously implanting the signal delivery device further includes directing the percutaneous insertion device out of the patient at a second location.

23. The method of example 22 wherein directing the percutaneous insertion device out of the patient at the second location includes directing the percutaneous insertion device out of the patient at an intraoral location.

24. The method of example 22 wherein directing the percutaneous insertion device out of the patient at the second location includes directing the percutaneous insertion device out of the patient at a submandibular location.

25. The method of any of examples 1-24 wherein the target signal delivery location includes a hypoglossal nerve, a medial branch of the hypoglossal nerve, an ansa cervicalis nerve, a genioglossus muscle, and/or a geniohyoid muscle of the patient.

26. The method of any of examples 1-25 further comprising:
coupling a first suture thread to a first end of the signal delivery device; and
coupling a second suture thread to a second end of the signal delivery device,
wherein percutaneously implanting the signal delivery device further includes selectively pulling at least one of the first suture thread or the second suture thread to position the signal delivery device at the target signal delivery location.

27. A method for treating a patient, comprising:
percutaneously implanting a signal delivery device alongside a medial branch of a hypoglossal nerve of the patent, wherein—
   the signal delivery device includes an electrode, and wherein the electrode is positioned inferior to the medial branch and inferior to a retruser extending from the medial branch, and/or
   the retruser extends away from the medial branch in a first area, and the electrode is positioned to deliver an electrical signal to a second area opposite from the first area; and
providing power to the electrode from a wearable power source to treat a sleep disorder of the patient.

28. The method of example 27 wherein providing the power includes transmitting the power to the electrodes via an RF link.

29. The method of example 28 wherein transmitting the power via the RF link includes transmitting the power at a frequency in a frequency range between 400 MHz and 2.5 GHz.

30. The method of example 29 wherein transmitting the power includes transmitting the power at a frequency in a frequency range between 900 MHz and 1.2 GHz.

31. The method of any of examples 27-30 wherein providing the power to the electrode includes causing the electrode to deliver an electrical signal having at least one of:
an interpulse delay between 10 μs and 250 μs;
a peak-to-peak amplitude between 0.5 mA and 12 mA; or
a frequency in a frequency range between 10 Hz and 500 Hz.

32. The method of example 31 wherein providing the power to the electrode includes causing the electrode to deliver an electrical signal to the patient without activating the retruser.

33. The method of any of examples 27-32, further comprising:
before implanting the signal delivery device, percutaneously inserting a needle into the patient along a trajectory toward the medial branch of the patient's hypoglossal nerve; and
aligning the needle with the medial branch, and wherein implanting the signal delivery device includes directing the signal delivery device along the trajectory of the needle.

34. The method of example 33 further comprising delivering an electrical signal to the patient via the needle to aid in positioning the signal delivery device.

35. The method of any of examples 27-34 wherein percutaneously implanting the signal delivery device includes percutaneously injecting the signal delivery into the patient at a submandibular location, an intraoral location, or a sublingual location.

36. The method of any of examples 27-35 wherein the signal delivery device is a first signal delivery device, the method further comprising percutaneously implanting a second signal delivery device proximate a target location.

37. The method of example 36 wherein the target location includes another hypoglossal nerve, a medial branch of the hypoglossal nerve, an ansa cervicalis nerve, a genioglossus muscle, and/or a geniohyoid muscle of the patient.

38. The method of example 36 or example 37 wherein the medial branch of the patient's hypoglossal nerve is a medial branch of a left hypoglossal nerve of the patient, and wherein percutaneously implanting the second signal delivery device proximate the target location includes percutaneously implanting the second signal delivery device proximate a medial branch of a right hypoglossal nerve of the patient.

39. The method of any of examples 27-38 wherein providing the power to treat the sleeping disorder includes inducing a motor response of the patient's tongue.

40. The method of example 39 wherein inducing the motor response in the patient's tongue includes at least one of causing the patient's tongue to move anteriorly away from the patient's airway or inducing caudal traction of the patient's hyoid bone and/or thyroid cartilage.

41. A signal delivery device, comprising:
a housing;
an antenna positioned within the housing and configured to receive a wireless power signal via a wearable power source;
a signal generator positioned within the housing and operably coupled to the antenna; and
an electrode carried by the housing and operably coupled to the signal generator, wherein the electrode extends at least partially around at least one of (1) at least a portion the signal generator or (2) at least a portion of the antenna.

42. The signal delivery device of example 41 wherein the housing includes a first housing portion and a second housing portion, wherein the electrode is positioned at an exterior surface of the first housing portion, wherein the signal generator is positioned within the first housing portion, and wherein the antenna is positioned within the second housing portion.

43. The signal delivery device of example 41 or example 42 wherein the signal generator includes circuitry and/or a charge pump, and wherein the electrode extends at least partially around the circuity and/or the charge pump.

44. The signal delivery device of any of examples 41-43 wherein the electrode is configured to be positioned inferior to a medial branch of a hypoglossal nerve of the patient and inferior to at least one retruser extending from the medial branch.

45. The signal delivery device of any of examples 41-44 wherein the signal generator is configured to cause the electrode to deliver an electrical signal, wherein the electrical signal has signal delivery parameters including:
an interpulse delay between 10 μs and 250 μs,
a peak-to-peak amplitude between 0.5 mA and 12 mA, and
a frequency in a frequency range between 10 Hz and 500 Hz.

46. The signal delivery device of any of examples 41-45 wherein the electrode is configured to apply an electrical signal to a target location of the patient without stimulating any retruser of the patient.

47. The signal delivery device of any of examples 41-46 wherein the one electrode is circumferentially masked or circumferentially segmented.

48. A system for delivering electrical signals to a patient, the system comprising:
a percutaneously-deliverable lead body having a plurality of electrodes, with individual electrodes connected to corresponding first terminals carried by the lead body; and
a separate, percutaneously-deliverable housing having second terminals positioned to couple with the first terminals during an implant procedure, the housing having a pulse generator coupled to the second terminals, and a power receiving antenna coupled to the pulse generator.

49. The system of example 48, wherein the percutaneously-deliverable lead body is configured to be positioned such that at least one of the plurality of electrodes is inferior to a medial branch of a hypoglossal nerve of the patient and inferior to at least one retruser extending from the medial branch.

50. The system of example 48 or example 49 wherein the pulse generator is configured to cause one or more of the plurality of electrodes to deliver an electrical signal to the medial branch, wherein the first electrical signal has first signal delivery parameters including:
an interpulse delay between 10 μs and 250 μs,
a peak-to-peak amplitude between 0.5 mA and 12 mA, and
a frequency in a frequency range between 10 Hz and 500 Hz.

51. The system of any of examples 48-50 wherein the percutaneously-deliverable lead body and the percutaneously-deliverable housing comprise a first implantable device, the system further comprising:
a second implantable device configured to be positioned proximate a target stimulation location of the patient and to deliver a second electrical signal to the target stimulation location,
wherein the target stimulation location includes another medial branch of another hypoglossal nerve of the patient, an ansa cervicalis nerve of the patient, a genioglossus muscle of the patient, and/or a geniohyoid muscle of the patient.

52. The system of example 51 wherein the first signal delivery device is configured to deliver a first electrical signal having one or more first signal delivery parameters, wherein the second signal delivery device is configured to deliver a second electrical signal having one or more second signal delivery parameters.

53. The system of example 52 wherein at least one of the one or more first signal delivery parameters has a different value than a corresponding one of the one or more second signal delivery parameters.

54. The system of example 52 or example 53 wherein the one or more first signal delivery parameters include a first amplitude, wherein the one or more second signal delivery parameters include a second amplitude, and wherein the second amplitude is different than the first amplitude.

55. The system of any of examples 48-54 wherein at least one of the plurality of electrodes is configured to apply an electrical signal to a target location of the patient without stimulating at least one retruser of the patient.

56. The system of example 55 wherein the at least one electrode is circumferentially masked or circumferentially segmented.

57. The system of any of examples 48-56 wherein—
the housing includes a connector housing, wherein the connector housing includes the second terminals and an axial lead body opening configured to releasably receive the first terminals of the lead body therethrough;
the first terminals are positioned on an outer surface of the lead body and configured to be positioned within a corresponding one of the second terminals via the axial lead body opening;
the lead body has a first outer diameter;
the housing has a second outer diameter greater than the first outer diameter; and
the power receiving antenna is configured to receive RF signals from a wearable power source.

58. A method for treating a patient, comprising:
percutaneously inserting a needle into the patient along a trajectory toward a medial branch of a hypoglossal nerve of the patient;
aligning the needle with the medial branch;
percutaneously implanting a signal delivery device alongside the medial branch via the trajectory defined by the needle, wherein the signal delivery device includes an electrode, and wherein the electrode is positioned to produce a net positive protrusive motor response of the patient's tongue, and wherein—
   the electrode is positioned inferior to the medial branch and inferior to a retruser extending from the medial branch, and/or
   the retruser extends away from the medial branch in a first area, and the electrode is positioned to deliver electrical stimulation to a second area opposite from the first area; and
providing power to the electrode from a wearable power source to treat a sleep disorder of the patient,
wherein—
   percutaneously inserting the needle includes directing the needle into the patient at a submandibular location or an intraoral location and delivering a first electrical signal to the patient via the needle;
   providing the power includes transmitting power to the electrodes via an RF link and causing the electrode to deliver a second electrical signal having at least one of:
      an interpulse delay between 10 μs and 250 μs,
      a peak-to-peak amplitude between 0.5 mA and 12 mA, or
      a first frequency in a first frequency range between 10 Hz and 500 Hz; and
   transmitting the power via the RF link includes transmitting the power at a second frequency in a second frequency range between 400 MHz and 2.5 GHz.

59. The method of example 58 wherein the signal delivery device is a first signal delivery device, the method further comprising percutaneously implanting a second signal delivery device proximate a target stimulation location

60. The method of example 59 wherein the target stimulation location includes another portion of a hypoglossal nerve of the patient, an ansa cervicalis nerve of the patient, a genioglossus muscle of the patient, and/or a geniohyoid muscle of the patient.

61. The method of example 59 or example 60 wherein the medial branch of the patient's hypoglossal nerve is a medial branch of a left hypoglossal nerve of the patient, and wherein percutaneously implanting the second signal delivery device proximate the target simulation location includes percutaneously implanting the second signal delivery device proximate a medial branch of a right hypoglossal nerve of the patient.

62. The method of any of examples 58-61 wherein the net positive protrusive response includes a retrusive response and a protrusive response greater than the retrusive response.

63. The method of any of examples 58-62 wherein providing the power to the electrode includes causing the electrode to deliver the electrical signal without activating any retruser of the patient.

64. The method of any of examples 58-63 wherein inducing the net positive protrusive motor response in the patient's tongue includes at least one of causing the patient's tongue to move anteriorly away from the patient's airway or inducing caudal traction of the patient's hyoid bone and/or thyroid cartilage.

65. The method of any of examples 58-64 wherein inducing the net positive protrusive motor response in the patient's tongue includes causing an airway of the patient to open or further open in response to delivery of the electrical signal.

I/we claim:

1. A method for treating a patient, comprising:
percutaneously inserting a needle into the patient along a trajectory toward a medial branch of a hypoglossal nerve of the patient;
aligning the needle with the medial branch;
percutaneously implanting a signal delivery device alongside the medial branch via the trajectory defined by the needle, wherein the signal delivery device includes an electrode, and wherein the electrode is positioned to produce a net positive protrusive motor response of the patient's tongue, and wherein—
the electrode is positioned inferior to the medial branch and inferior to a retruser extending from the medial branch, and/or
the retruser extends away from the medial branch in a first area, and the electrode is positioned to deliver electrical stimulation to a second area opposite from the first area; and
providing power to the electrode from a wearable power source to treat a sleep disorder of the patient,
wherein—
percutaneously inserting the needle includes directing the needle into the patient at a submandibular location or an intraoral location and delivering a first electrical signal to the patient via the needle;
providing the power includes transmitting power to the electrode via an RF link and causing the electrode to deliver a second electrical signal having at least one of:
an interpulse delay between 10 μs and 250 μs,
a peak-to-peak amplitude between 0.5 mA and 12 mA, or
a first frequency in a first frequency range between 10 Hz and 500 Hz; and
transmitting the power via the RF link includes transmitting the power at a second frequency in a second frequency range between 400 MHz and 2.5 GHz.

2. The method of claim 1 wherein the signal delivery device is a first signal delivery device, the method further comprising percutaneously implanting a second signal delivery device proximate a target location.

3. The method of claim 2 wherein the target location includes another hypoglossal nerve of the patient, an ansa cervicalis nerve of the patient, a genioglossus muscle of the patient, and/or a geniohyoid muscle of the patient.

4. The method of claim 2 wherein the medial branch of the patient's hypoglossal nerve is a medial branch of a left hypoglossal nerve of the patient, and wherein percutaneously implanting the second signal delivery device proximate the target location includes percutaneously implanting the second signal delivery device proximate a medial branch of a right hypoglossal nerve of the patient.

5. The method of claim 1 wherein the net positive protrusive motor response includes a retrusive response and a protrusive response greater than the retrusive response.

6. The method of claim 1 wherein providing the power to the electrode includes causing the electrode to deliver the second electrical signal without activating any retruser of the patient.

7. The method of claim 1 wherein inducing the net positive protrusive motor response in the patient's tongue includes at least one of causing the patient's tongue to move anteriorly away from the patient's airway or inducing caudal traction of the patient's hyoid bone and/or thyroid cartilage.

8. The method of claim 1 wherein inducing the net positive protrusive motor response in the patient's tongue includes causing an airway of the patient to open or further open in response to delivery of the second electrical signal.

9. A method for treating a patient, comprising:
percutaneously implanting a signal delivery device at a target signal delivery location in the patient, wherein the signal delivery device includes an electrode, and wherein the electrode is positioned to produce a net positive protrusive motor response of the patient's tongue; and
providing power to the electrode from a wearable power source to cause the electrode to deliver an electrical signal to the target signal delivery location to produce the net positive protrusive motor response.

10. The method of claim 9 wherein percutaneously implanting the signal delivery device includes percutaneously implanting the signal delivery device alongside a medial branch of a patient's hypoglossal nerve, and wherein the electrode is positioned inferior to the medial branch and inferior to at least one retruser extending from the medial branch.

11. The method of claim 9 wherein percutaneously implanting the signal delivery device includes percutaneously implanting the signal delivery device alongside a medial branch of a hypoglossal nerve of the patient, wherein the medial branch includes a retruser extending away from the medial branch in a first area, and wherein the electrode is positioned to deliver electrical stimulation to a second area opposite from the first area.

12. The method of claim 9 wherein the net positive protrusive motor response includes a retrusive response and a protrusive response greater than the retrusive response.

13. The method of claim 9 wherein providing the power to the electrode includes causing the electrode to deliver the electrical signal without activating any retruser of the patient.

14. The method of claim 9 wherein inducing the net positive protrusive motor response of the patient's tongue includes at least one of causing the patient's tongue to move anteriorly away from the patient's airway or inducing caudal traction of the patient's hyoid bone and/or thyroid cartilage.

15. The method of claim 9 wherein the target signal delivery location includes a hypoglossal nerve, an ansa cervicalis nerve, a genioglossus muscle, a geniohyoid muscle, a sternohyoid muscle, a thyrohyoid muscle, an omohyoid muscle, and/or a sternothyroid muscle of the patient.

16. The method of claim 9 wherein inducing the net positive protrusive motor response in the patient's tongue includes causing an airway of the patient to open or further open in response to delivery of the electrical signal.

17. The method of claim 9 wherein the signal delivery device is a first signal delivery device and the target signal delivery location is a first target signal delivery location, the method further comprising percutaneously implanting a second signal delivery device proximate a second target signal delivery location.

18. The method of claim 9 wherein providing the power includes transmitting power to the electrode via an RF link.

19. The method of claim 18 wherein transmitting the power via the RF link includes transmitting the power at a frequency in a frequency range between 400 MHz and 2.5 GHz.

20. The method of claim 18 wherein transmitting the power includes transmitting the power at a frequency in a frequency range between 900 MHz and 1.2 GHz.

21. The method of claim 9 wherein providing the power to the electrode includes causing the electrode to deliver an electrical signal having at least one of:
an interpulse delay between 10 μs and 250 μs;
a peak-to-peak amplitude between 0.5 mA and 12 mA; or
a frequency in a frequency range between 10 Hz and 500 Hz.

22. The method of claim 9 wherein the electrode a plurality of circumferential segments and wherein providing the power to the electrode includes causing the electrode to deliver the electrical signal via individual ones of the circumferential segments of the electrode.

23. The method of claim 9, further comprising:
before implanting the signal delivery device, percutaneously inserting a needle into the patient along a trajectory toward a medial branch of the patient's hypoglossal nerve; and
aligning the needle with the medial branch, wherein implanting the signal delivery device includes directing the signal delivery device along the trajectory of the needle.

24. The method of claim 23 wherein the electrical signal is a first electrical signal, the method further comprising delivering a second electrical signal to the patient via the needle to aid in positioning the signal delivery device.

25. The method of claim 24 wherein the first electrical signal has a first signal delivery parameter, and wherein the second electrical signal has a second signal delivery parameter different than the first signal delivery parameter.

26. The method of claim 9 wherein percutaneously implanting the signal delivery device includes directing a percutaneous insertion device into the patient at a first location.

27. The method of claim 26 wherein directing the percutaneous insertion device into the patient at the first location includes directing the percutaneous insertion device into the patient at a submandibular location.

28. The method of claim 26 wherein directing the percutaneous insertion device into the patient at the first location includes directing the percutaneous insertion device into the patient at an intraoral location.

29. The method of claim 28 wherein directing the percutaneous insertion device into the patient at the intraoral location includes directing the percutaneous insertion device into the patient at a sublingual location.

30. The method of claim 26 wherein percutaneously implanting the signal delivery device further includes directing the percutaneous insertion device out of the patient at a second location.

31. The method of claim 30 wherein directing the percutaneous insertion device out of the patient at the second location includes directing the percutaneous insertion device out of the patient at an intraoral location.

32. The method of claim 30 wherein directing the percutaneous insertion device out of the patient at the second location includes directing the percutaneous insertion device out of the patient at a submandibular location.

33. The method of claim 9 wherein the target signal delivery location includes a hypoglossal nerve, a medial branch of the hypoglossal nerve, an ansa cervicalis nerve, a genioglossus muscle, and/or a geniohyoid muscle of the patient.

34. The method of claim 9 further comprising:
coupling a first suture thread to a first end of the signal delivery device; and
coupling a second suture thread to a second end of the signal delivery device,
wherein percutaneously implanting the signal delivery device further includes selectively pulling at least one of the first suture thread or the second suture thread to position the signal delivery device at the target signal delivery location.

35. A signal delivery device, comprising:
a housing;
an antenna positioned within the housing and configured to receive a wireless power signal via a wearable power source;
a signal generator positioned within the housing and operably coupled to the antenna; and
an electrode carried by the housing and operably coupled to the signal generator, wherein the electrode extends at least partially around at least one of (1) at least a portion the signal generator or (2) at least a portion of the antenna.

36. The signal delivery device of claim 35 wherein the housing includes a first housing portion and a second housing portion, wherein the electrode is positioned at an exterior surface of the first housing portion, wherein the signal generator is positioned within the first housing portion, and wherein the antenna is positioned within the second housing portion.

37. The signal delivery device of claim 35 wherein the signal generator includes circuitry and/or a charge pump, and wherein the electrode extends at least partially around the circuity and/or the charge pump.

38. The signal delivery device of claim 35 wherein the electrode is configured to be positioned inferior to a medial branch of a hypoglossal nerve of a patient and inferior to at least one retruser extending from the medial branch.

39. The signal delivery device of claim 35 wherein the signal generator is configured to cause the electrode to deliver an electrical signal, wherein the electrical signal has signal delivery parameters including:
an interpulse delay between 10 μs and 250 μs,
a peak-to-peak amplitude between 0.5 mA and 12 mA, and
a frequency in a frequency range between 10 Hz and 500 Hz.

40. The signal delivery device of claim 35 wherein the electrode is configured to apply an electrical signal to a target location of a patient without stimulating any retruser of the patient.

41. The signal delivery device of claim 35 wherein the one electrode is circumferentially masked or circumferentially segmented.
